(19) United States
(12) Patent Application Publication
(10) Pub. No.: US 2006/0141475 A1
(43) Pub. Date: Jun. 29, 2006

(54) Polynucleotide sequence detection assays and analysis

(75) Inventors: Barnett Rosenblum, San Jose, CA (US); David P. Holden, Burlingame, CA (US); Yuandan Lou, Cupertino, CA (US); Ryan T. Koehler, Hayward, CA (US); Srinivasa Ramachandra, Danville, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 10/718,237

(22) Filed: Nov. 19, 2003

Related U.S. Application Data
(60) Provisional application No. 60/427,818, filed on Nov. 19, 2002.
(60) Provisional application No. 60/445,636, filed on Feb. 7, 2003.
(60) Provisional application No. 60/445,494, filed on Feb. 7, 2003.

(51) Int. Cl. C12Q 1/68; G06F 19/00; G01N 33/53

(52) U.S. Cl. 435/6; 702/20; 435/7.1; 436/86

(57) ABSTRACT

Methods and software for associating mobility probes with target macromolecules are discussed. By encoding the identities of macromolecules of interest with a universal set of tag portions complementary to a universal set of mobility probes, reactions varying in their input starting material may be identified using the same universal set of mobility probes. This allows the universal collection of mobility probes to be used in a target macromolecule-independent manner. Software is used to decode the associations between the mobility probes and a given target macromolecular identity.

### CROSS-REFERENCE TO RELATED APPLICATIONS

This application claims the benefit under 35 USC §119(e) of (1) U.S. Provisional Patent Application Ser. No. 60/427818, filed Nov. 19, 2002, (2) U.S. Provisional Patent Application Ser. No. 60/445636, filed Feb. 7, 2003, (3) U.S. Provisional Patent Application Ser. No. 60/445494, filed Feb. 7, 2003, all of which are assigned to the assignee hereof, and all of which are expressly incorporated herein by reference in their entireties. Attorney Docket No. 4992US, entitled, “Polynucleotide Sequence Detection Assays” filed Nov. 19, 2003 which is assigned to the assignee hereof is expressly incorporated herein by reference in its entirety.

### FIELD OF THE INVENTION

The present invention relates to methods for detecting one or more polynucleotide sequences in one or more samples, to reagents and kits for use therein, and to methods of analysis related thereto.

### INTRODUCTION

Methods for detection and analysis of target nucleic acids have found wide utility in basic research, clinical diagnostics, forensics, and other areas. One important use is in the area of genetic polymorphism. Genetic polymorphisms generally concern the genetic sequence variations that exist among homologous loci from different members of a species. Genetic polymorphisms can arise through the mutation of genetic loci by a variety of processes, such as errors in DNA replication or repair, genetic recombination, spontaneous mutations, transpositions, etc. Such mutations can result in single or multiple base substitutions, deletions, or insertions, as well as transpositions, duplications, etc.

Single base substitutions (transitions and transversions) within gene sequences can cause missense mutations and nonsense mutations. In missense mutations, an amino acid residue is replaced by a different amino acid residue, whereas in nonsense mutations, stop codons are created that lead to truncated polypeptide products. Mutations that occur within signal sequences, e.g., for directing exon/intron splicing of mRNAs, can produce defective splice variants with dramatically altered protein sequences. Deletions, insertions, and other mutations can also cause frameshifts in which contiguous residues encoded downstream of the mutation are replaced with entirely different amino acid residues. Mutations outside of exons can interfere with gene expression and other processes.

Genetic mutations underlie many disease states and disorders. Some diseases have been traced directly to single point mutations in genomic sequences (e.g., the A to T mutation associated with sickle cell anemia), while others have been correlated with large numbers of different possible polymorphisms located in the same or different genetic loci (e.g., cystic fibrosis). Mutations within the same genetic locus can produce different diseases (e.g., hemoglobinopathies). In other cases, the presence of a mutation may indicate susceptibility to particular condition for a disease but is insufficient to reliably predict the occurrence of the disease with certainty. Most known mutations have been localized to gene-coding sequences, splice signals, and regulatory sequences. However, it is expected that mutations in other types of sequences can also lead to deleterious, or sometimes beneficial, effects.

The large number of potential genetic polymorphisms poses a significant challenge to the development of methods for identifying and characterizing nucleic acid samples and for diagnosing and predicting disease. In other applications, it is desirable to detect the presence of pathogens or exogenous nucleic acids and to detect or quantify RNA transcript levels.

In light of the increasing amount of sequence data that is becoming available for various organisms, and particularly for higher organisms such as humans, there is a need for rapid and convenient methods for determining the presence or absence of allelic variants, such as single nucleotide polymorphisms, and target mutations. Ideally, such a method should have high sensitivity, accuracy, and reproducibility. Also, the method should allow simultaneous detection of multiple target sequences in a single reaction mixture.

### SUMMARY

The present invention, in some embodiments, provides a method for detecting at least one target sequence in a sample. In the method, a sample that contains, or may contain, a plurality of target sequences is combined with a plurality of different probe sets. Each probe set comprises (a) a first probe comprising a first target-specific portion and a 5′ primer-specific portion, and (b) a second probe comprising a second target-specific portion and a 3′ primer-specific portion, wherein the first and second probes in each set are suitable for ligation together when hybridized to adjacent complementary target sequences. The first or second probe in each set further comprises an identifier tag portion that is between the primer-specific portion and the target-specific portion. The identifier tag portion identifies the probe that contains the identifier tag portion.

The ligation reaction mixture is subjected to at least one cycle of ligation, wherein adjacently hybridized first and second probes of at least one probe set are ligated together to form a ligation product comprising a 5′ primer-specific portion, first and second target-specific portions, a 3′ primer-specific portion, and an identifier tag portion, to form a first strand.

In some embodiments, the first or second probe comprises an affinity moiety, such as biotin, for use in a solid-phase separation step. For example, the second probe can comprise an affinity moiety at its 3′ end, so that the resulting ligation product can be captured by a support-bound affinity partner, such as streptavidin, to allow non-ligation components of the reaction mixture to be washed away. Alternatively, in another non-limiting example, the first probe may comprise an affinity moiety at its 5′ end. In some embodiments, capture is performed prior to ligation. In other embodiments, capture is performed after ligation, before amplification.

In other embodiments, unligated probes can be selectively degraded after ligation by exonuclease treatment to cleave unligated probes. For example, in one non-limiting example, a 5′ single-strand specific exonuclease can be used to cleave residual second probes, whereas ligation products are protected from 5′ exonuclease degradation due to the absence of a free 5′ phosphate group at the 5′ end of the first probes (and 5′ end of the ligation product).

In some embodiments, the first strand from the ligation reaction is combined with a reverse primer that is complementary to the 3′ primer-specific portion, and the primer is extended with a polymerase to form a double-stranded product comprising the first strand and a complementary, second strand that is hybridized to the first strand.

In some embodiments, the first strand, the second strands, or both, are amplified by polymerase-mediated extension of a forward primer and/or the reverse primer, wherein the first primer is complementary to the complement of said 5′ primer-specific portion, to form amplified first strand and/or second strands.

Following amplification, one or more complexes are formed, wherein each complex comprises an amplified strand and a mobility probe. The mobility probe comprises (a) a mobility defining moiety that imparts an identifying mobility or total mass to the mobility probe, and (b) a tag portion or tag portion complement, and wherein the tag portion or tag portion complement is hybridized to the complementary tag portion complement or tag portion, respectively, in the amplified strand. For detection by spectrophotometric methods, for example, (e.g., fluorescence detection), the mobility probe may additionally comprise (c) a detectable label, such as a fluorescent label.

In some embodiments, the complex is captured on a solid support. For this purpose, the first probe, second probe, first primer, or second primer may include an affinity moiety (which may be the same or different from the affinity moiety mentioned above in connection with ligation, if used), and the solid support comprises an affinity moiety binding partner. After an amplified strand is captured on the solid support, the support may be washed to remove undesired reaction components. In other embodiments, undesired reaction components may be removed by size exclusion chromatography, ultrafiltration, exonuclease treatment, or other technique, with or without using an affinity capture step.

Following complex formation, and optional affinity capture and washing, one or more mobility probes are released from the one or more complexes and are detected by a mobility-dependent analysis technique (MDAT), such as electrophoresis, chromatography, or mass spectrometry. From the presence or absence of a particular mobility probe, as evidenced by its particular mobility observed by the MDAT, the presence or absence of each target sequence can be determined.

In some embodiments, a probe set is used that comprises a probe that contains a T nucleotide at a selected position to detect conversion of cytosine to uracil (indicating that the cytosine was not methylated), and a second probe that contains an A nucleotide at the selected position to detect conversion of cytosine to thymine (indicating that the cytosine was methylated). The relative amounts of T versus A that are detected can also be used to estimate the average amount of methylation for one or more particular cytosine nucleotides.

More broadly, the invention also includes methods as described above, but wherein the amplification step is optional, and the ligation step optionally includes one or more cycles of ligase chain reaction to increase the amount of ligation product (and its complement). For ligase chain reaction, the probe sets comprise third and fourth probes that are complementary to the target-specific portions of the first and second probes, respectively, to generate one or more copies of the complementary strand of the first ligation product. Thus, for such embodiments, primer-specific portions are unnecessary and can be omitted from the probes. Following ligation and optional additional cycles of probe ligation, the ligation products and/or their complements can be combined with mobility probes as above, to form complexes that comprise a ligation product (single or double stranded) and a mobility probe which are hybridized together via the complementary tag portion and tag portion complement. The mobility probes may then be released and detected, to determine the presence or absence of the target sequences.

The invention contemplates the use of mobility probes as a general approach for determining (e.g., elucidating the identity, presence, absence, etc. of) any macromolecule in a sample or complex plurality. In one such embodiment, the tag portions are attached to a protein library in such a way as to associate a particular protein with the eventual mobility probe that will hybridize to the tag portion. By contacting a tag portion labeled protein library with a prospective affinity partner, and washing away unbound library proteins, followed by hybridizing a library of mobility probes to the tag portions and washing away unbound mobility probes, the identity of the protein bound to the affinity partner can be elucidated by virtue of the identity of the eluted mobility probe. Representative macromolecular determinations contemplated by the instant invention include, but are not limited to, nucleic acids, proteins, lipids, carbohydrates, glycoproteins, and drug-receptor interactions (generally referred to, collectively, as biochemicals or biochemical complexes).

The present teachings also contemplate software adapted to perform the association and deconvolution between a particular mobility probe and a particular macromolecule. By encoding the respective identities of a plurality of macromolecules with a universal set of tag portions complementary to a universal set of mobility probes, reactions varying in their input starting material may be identified by the same universal set of mobility probes, thus allowing the universal collection of mobility probes to be used in a target macromolecule-independent manner. Indeed, a benefit of various embodiments of the instant invention involves the cost savings and economies of scale afforded by this universal mobility probe set coupled with a common MDAT readout platform. An aspect of this software can be to associate, in a reaction-specific context, the pairing of a given mobility probe with a given target macromolecular identity. Because reactions possessing different candidate binding partners yet identical temperature requirements can be performed in parallel in different wells of a microtiter plate, or the like, an aspect of the data analyses can include underlying algorithms to associate a given mobility probe in one well with a given macromolecular identity, while associating that same mobility probe in a different well with a different macromolecular identity. For example, in a SNP detection experiment, the pseudo-code can convey:

If well=A1,

Then Mobility Probe X=Alpha allele,

Mobility Probe Y=Beta allele
For Mobility Probe X, Print “Alpha allele homozygote”
For Mobility Probe Y, Print “Beta allele Heterozygote”
For Mobility Probe X and Y, Print “Alpha/Beta Heterozygote”

Else, If well=A2

Then Mobility Probe X=Delta allele,

Mobility Probe Y=Epsilon allele

For Mobility Probe X, Print Delta allele homozygote
For Mobility Probe Y, Print Epsilon allele homozygote
For Mobility Probe X and Y, Print Delta/Epsilon Heterozygote

Whereas in a different experimental design, with different macromolecules under investigation:

If well=A1,

Then Mobility Probe X=Protein A,

Mobility Probe Y=Protein B

For Mobility Probe X, Print “Protein A”
For Mobility Probe Y, Print “Protein B”

Else, If well=A2

Mobility Probe X=Protein C

Mobility Probe Y=Protein D

For Mobility Probe X, Print “Protein C”
For Mobility Probe Y, Print “Protein D”

Also provided are reagents and kits, which may be useful in practicing various methods of the invention.

These and other features and advantages of the invention will become more readily apparent in light of the detailed description herein.

### BRIEF DESCRIPTION OF THE DRAWINGS

FIG. 1 illustrates an exemplary probe set in accordance with some embodiments of the invention.

FIG. 2 illustrates a way to differentiate between two potential alleles in a target locus by ligation, in accordance with certain embodiments of the invention.

FIG. 3 illustrates an exemplary scheme in accordance with some embodiments of the invention.

FIG. 4 illustrates another exemplary scheme in accordance with some embodiments of the invention.

FIG. 5 shows a simplified electropherogram of several mobility probe peaks.

FIG. 6 illustrates an exemplary instrument system for detection of the mobility probes in accordance with some embodiments of the invention.

FIG. 7 illustrates an exemplary set of processing sets used to relate features of mobility data to the presence r absence of target biochemicals or biochemical probes.

FIG. 8 illustrates an exemplary system for analyzing mobility data generated by an electropherogram.

FIG. 9 illustrates an exemplary system for making allele calls.

FIG. 10 is a block diagram of a computer system that is in accordance with some embodiments of the invention.

FIG. 11 illustrates the process of binning electropherogram data, in accordance with some embodiments of the invention.

FIG. 12 illustrates an exemplary allele caller which makes use of peak ratios.

FIG. 13 illustrates the benefit of performing cluster analysis. FIG. 13(a) shows unclustered data. FIG. 13(b) shows data clustered in the peak height space. FIG. 13(c) shows data clustered in the rho/Theta space.

FIG. 14 outlines the steps in an exemplary clustering algorithm

### DETAILED DESCRIPTION

The present invention provides methods for detecting one or more selected target polynucleotide sequences in a sample. The invention permits detection of target sequences with high specificity and sensitivity, allowing detection and/or quantitation of small amounts of target sequences. In some embodiments, the invention is also advantageous for genotyping and detection of genetic polymorphisms.

Definitions

It is to be understood that both the foregoing general description and the following detailed description are exemplary and explanatory only and are not restrictive of the invention. In this application, the use of the singular includes the plural unless specifically stated otherwise. For example, “a probe” means that more than one probe may be present. Also, the use of “or” means “and/or” unless stated otherwise. Similarly, “comprise”, “comprises”, “comprises”, “include”, “includes”, and “including” are not intended to be limiting.

The term “nucleoside” refers to a compound comprising a purine, deazapurine, or pyrimidine nucleobase, e.g., adenine, guanine, cytosine, uracil, thymine, 7-deazaadenine, 7-deazaguanosine, and the like, that is linked to a pentose at the 1′-position. When the nucleoside base is purine or 7-deazapurine, the pentose is attached to the nucleobase at the 9-position of the purine or deazapurine, and when the nucleobase is pyrimidine, the pentose is attached to the nucleobase at the 1-position of the pyrimidine.

The term “nucleotide” as used herein refers to a phosphate ester of a nucleoside, e.g., a triphosphate ester, wherein the most common site of esterification is the hydroxyl group attached to the C-5 position of the pentose. See, e.g., Kornberg and Baker, DNA Replication, 2nd Ed. (Freeman, San Francisco, 1992).

The term “polynucleotide” means polymers of nucleotide monomers, including analogs of such polymers, including double- and single-stranded deoxyribonucleotides, ribonucleotides, ?-anomeric forms thereof, and the like. Monomers are linked by “intemucleotide linkages,” e.g., phosphodiester linkages, where as used herein, the term “phosphodiester linkage” refers to phosphodiester bonds or bonds including phosphate analogs thereof, including associated counterions, e.g., H⁺, NH₄+, Na⁺, if such counterions are present. Whenever a polynucleotide is represented by a sequence of letters, such as “ATGCCTG,” it will be understood that: (i) the nucleotides are in 5′ to 3′ order from left to right “ATGCCTG,” it will be understood that: (i) the nucleotides are in 5′ to 3′ order from left to right was intended; and (ii) that “A” denotes deoxyadenosine, “C” denotes deoxycytidine, “G” denotes was intended; and (ii) that “A” denotes deoxyadenosine, “C” denotes deoxycytidine, “G” denotes oligonucleotides can be found, among other places, in U.S. Pat. Nos. 4,373,071; 4,401,796; 4,415,732; 4,458,066; 4,500,707; 4,668,777; 4,973,679; 5,047,524; 5,132,418; 5,153,319; and 5,262,530.

“Analogs” in reference to nucleosides and/or polynucleotides comprise synthetic analogs having modified nucleobase portions, modified pentose portions and/or modified phosphate portions, and, in the case of polynucleotides, modified intemucleotide linkages, as described generally elsewhere (e.g., Scheit, Nucleotide Analogs (John Wiley, N.Y., (1980); Englisch, Angew. Chem. Int. Ed. Engl. 30:613-29 (1991); Agrawal, Protocols for Polynucleotides and Analogs, Humana Press (1994)). Generally, modified phosphate portions comprise analogs of phosphate wherein the phosphorous atom is in the +5 oxidation state and one or more of the oxygen atoms is replaced with a non-oxygen moiety, e.g., sulfur. Exemplary phosphate analogs include but are not limited to phosphorothioate, phosphorodithioate, phosphoroselenoate, phosphorodiselenoate, phosphoroanilothioate, phosphoranilidate, phosphoramidate, boronophosphates, including associated counterions, if such counterions are present. Exemplary modified nucleobase portions include but are not limited to 2,6-diaminopurine, hypoxanthine, pseudouridine, C-5-propyne, isocytosine, isoguanine, 2-thiopyrimidine, and other like analogs. According to some embodiments, nucleobase analogs are iso-C and iso-G nucleobase analogs available from Sulfonics, Inc., Alachua, Fla. (e.g., Benner, et al., U.S. Pat. No. 5,432,272) or LNA analogs (e.g., Koshkin et al., Tetrahedron 54:3607-30 (1998)). Exemplary modified pentose portions include but are not limited to 2′- or 3′-modifications where the 2′- or 3′-position is hydrogen, hydroxy, alkoxy, e.g., methoxy, ethoxy, allyloxy, isopropoxy, butoxy, isobutoxy and phenoxy, azido, amino or alkylamino, fluoro, chloro, bromo and the like. Modified intemucleotide linkages include, but are not limited to, phosphate analogs, analogs having achiral and uncharged intersubunit linkages (e.g., Sterchak, E. P., et al., Organic Chem, 52:4202 (1987)), and uncharged morpholino-based polymers having achiral intersubunit linkages (e.g., U.S. Pat. No. 5,034,506). Intemucleotide linkage analogs include, but are not limited to,peptide nucleic acid (PNA), morpholidate, acetal, and polyamide-linked heterocycles. In some embodiments, one may use a class of polynucleotide analogs where a conventional sugar and intemucleotide linkage has been replaced with a 2-aminoethylglycine amide backbone polymer is PNA (e.g., Nielsen et al., Science, 254:1497-1500 (1991); Egholm et al., J. Am. Chem. Soc., 114: 1895-1897 (1992)).

A “target” or “target nucleic acid sequence” according to the present invention comprises a specific nucleic acid sequence that is to be detected and quantified. The term target nucleic acid sequence encompasses both DNA, RNA, and any analog thereof that has the ability to form base-paired duplexes or triplexes. The person of ordinary skill will appreciate that while the target nucleic acid sequence may be described as a single-stranded molecule, the complement of that single-stranded molecule, or a double-stranded target nucleic acid molecule may also serve as a target nucleic acid sequence. In addition, a target nucleic acid sequence may be the actual target nucleic acid present in a sample, or it may be a counterpart of that sequence, such as a cDNA derived from a target RNA sequence present in the starting material. In some embodiments, the target nucleic acid sequence may comprise single- or double-stranded DNA; cDNA, either single-stranded or double-stranded (e.g., DNA:DNA and DNA:RNA hybrids); and RNA, including, but not limited to, mRNA, mRNA precursors, and rRNA.

As used herein, “detecting” encompasses detection, quantification, and/or identification.

The term “amplification product” as used herein refers to the product of an amplification reaction including, but not limited to, primer extension, the polymerase chain reaction, RNA transcription, and the like. Thus, exemplary amplification products may comprise primer extension products, PCR amplicons, RNA transcription products, and/or the like.

Sample

The target nucleic acids for use with the invention may be derived from any organism or other source, including but not limited to prokaryotes, eukaryotes, plants, animals, and viruses, as well as synthetic nucleic acids, for example. Target nucleic acids may originate from any of a wide variety of sample types, such as cell nuclei (e.g., genomic DNA), whole cells, tissue samples, phage, plasmids, mitrochondria (containing MDNA), and the like. To reduce viscosity or improve hybridization kinetics, target nucleic acids may be sheared prior to use in the invention.

Many methods are available for the isolation and purification of target nucleic acids. Preferably, the target nucleic acids are sufficiently free of proteins and any other interfering substances to allow adequate target-specific probe annealing, cleavage, and ligation. Exemplary purification methods include (i) organic extraction followed by ethanol precipitation, e.g., using a phenol/chloroform organic reagent (Ausubel et al., eds., Current Protocols in Molecular Biology Vol. 1, Chapter 2, Section I, John Wiley & Sons, New York (1993)), preferably with an automated DNA extractor, e.g., a Model 341 DNA Extractor available from PE Applied Biosystems (Foster City, Calif.); (ii) solid phase adsorption methods (Walsh et al., Biotechnigues 10(4): 506-513, 1991; Boom et al., U.S. Pat. No. 5,234,809); and (iii) salt-induced DNA precipitation methods (Miller et al., Nucleic Acids Res. 16(3):9-10, 1988), such methods being typically referred to as “salting-out” methods. Optimally, each of the above purification methods is preceded by an enzyme digestion step to help eliminate protein from the sample, e.g., digestion with proteinase K, or other proteases.

To facilitate detection, the target nucleic acid can be amplified using a suitable amplification procedure prior to the ligation and amplification steps of various embodiments of the invention. Such amplification may be linear or exponential. In one embodiment, amplification of the target nucleic acid is accomplished using the polymerase chain reaction (PCR) (e.g., Mullis et al., eds, The Polymerase Chain Reaction, BirkHauser, Boston, Mass., 1994). Generally, the PCR consists of an initial denaturation step which separates the strands of a double stranded nucleic acid sample, followed by repetition of (i) an annealing step, which allows amplification primers to anneal specifically to positions flanking a target sequence; (ii) an extension step which extends the primers in a 5′ to 3′ direction thereby forming an amplicon nucleic acid complementary to the target sequence, and (iii) a denaturation step which causes the separation of the amplicon from the target sequence. Each of the above steps may be conducted at a different temperature, preferably using an automated thermocycler (Applied Biosystems, Foster City, Calif.).

If desired, RNA samples can be converted to DNA/RNA heteroduplexes or to duplex cDNA by known methods (e.g., Ausubel et al., supra; and Sambrook et al., Molecular Cloning: A Laboratory Manual, 2nd Edition, Cold Spring Harbor Laboratory, New York (1989); Sambrook and Russell, Molecular Cloning, Third Edition, Cold Spring Harbor Press (2000)). In addition, preparation of target nucleic acids can be accomplished using whole genome amplification techniques (e.g., Lizardi, U.S. Pat. No. 6,124,120).

In some embodiments, target nucleic acids are chemically treated prior to analysis. For example, analysis of the methylation state of cytosines can be performed using bisulfite as a modifying agent (e.g., see U.S. Pat. Nos. 6,265,171 and 6,331,393). Incubating target nucleic acid sequence with bisulfate results in deamination of a substantial portion of unmethylated cytosines, which converts such cytosines to uracil. Methylated cytosines are deaminated to a measurably lesser extent. In some embodiments, the sample is then amplified or replicated, resulting in the uracil bases being replaced with thymine. Thus, in some embodiments, a substantial portion of unmethylated target cytosines ultimately become thymines, while a substantial portion of methylated cytosines remain cytosines. In some embodiments, the identity of the nucleotide (cytosine, uracil, or thymine) of the target may be determined by a ligation and amplification method of the present invention, wherein a probe set is designed to detect the presence of either uracil or thymine at a known cytosine position in a bisulfite-treated target nucleic acid. In another embodiment, a probe set is used that comprises a probe that contains a T nucleotide at a selected position to detect conversion of cytosine to uracil (indicating that the cytosine was not methylated), and a second probe that contains an A nucleotide at the selected position to detect conversion of cytosine to thymine (indicating that the cytosine was methylated). The relative amounts of T versus A that are detected can also be used to estimate the average amount of methylation for one or more particular cytosine nucleotides.

Exemplary Reagents

The present invention employs probes that are designed to hybridize to complementary target sequences, and which are capable of undergoing ligation when hybridized to adjacent complementary regions in a target sequence. In some embodiments, probes of the invention can be used, for example, in linear and/or exponential probe ligation methods described herein.

Different probe sets can be prepared, wherein each probe set comprises at least a first probe and a second probe. The first probe comprises a first target-specific portion and a 5′ primer-specific portion, and the second probe comprises a second target-specific portion and a 3′ primer-specific portion. The first probe and the second probe in each set are designed to be suitable for ligation of the first target-specific portion to the second target specific portion when the first and second target-specific portions are hybridized to adjacent complementary target sequences. For example, the first probe can be designed such that the first target-specific portion is located on the 3′ end of the first probe, and the second probe can be designed so that the second target-specific portion is located on the 5′ end of the second probe. When the first and second probe are hybridized to adjacent complementary regions in the target sequence (adjacent target regions), the 3′ end of the first probe can be ligated to the 5′ end of the second probe.

The length of the target-specific portion in each probe is selected to ensure specific hybridization of the probe to the desired target sequence, without significant cross-hybridization to non-target nucleic acids. Also, to enhance binding specificity, the melting temperatures of the target-specific portions can be selected to be within a few degrees of each other. In some embodiments, the melting temperatures (Tm) of the target-specific portions are within a ΔTm range (Tmax−Tmin) of 10° C. or less, 5° C. or less, 3° C. or less, or 2° C. or less. This can be accomplished by suitable choice of sequence lengths for target-specific portions based on known methods for predicting melting temperatures (Breslauer et al., Proc. Natl. Acad. Sci 83:3746-3750 (1986); Rychlik et al., Nucleic Acids Res. 17:8543-8551 (1989) and 18:6409-6412 (1990); Wetmur, Crit. Rev. Biochem. Mol. Biol. 26:227-259 (1991); Osborne, CABIOS 8:83 (1991); Montpetit et al., J. Virol. Methods 36:119-128 (1992); and Kwok et al., Nucl. Acid Res. 18:999-1005, 1990), for example. See also Zuker et al, Algorithms and Thermodynamics for RNA Secondary Structure Prediction: A Practical Guide, in RNA Biochemistry and Biotechnology, pages 11-43, J. Barciszewski & B. F. C. Clark, eds., NATO ASI Series, Kluwer Academic Publishers (1999). Also, Version 3.0 of mfold for Unix operating systems is available via a free license for academic and nonprofit use only; commercial use is available for a fee. Copyright© is held by Washington University. Target-specific portions having lengths from 12 to 35 bases, 15 to 30 bases, or from 16 to 24 bases, for example, tend to be very sequence-specific when the annealing temperature is set within a few degrees of a probe melting temperature (Dieffenbach et al., in PCR Primer: A Laboratory Manual, Dieffenbach and Dveksler, eds., pp. 133-142, CSHL Press, New York (1995)). However, longer or shorter sequences can also be used. Also, when nucleotide analogs that have higher binding affinities for complementary nucleotides are included in a probe sequence (e.g., locked-nucleic acids), a shorter probe sequence can be used to achieve a particular Tm.

In some embodiments, the primer-specific portion in each probe can be designed to facilitate amplification of the ligation product (both the sense strand and the antisense strand) by allowing hybridization of a complementary primer for primer extension. The 3′ primer-specific portion, which is located downstream of (3′ relative to) the second target-specific portion in the second probe, can serve as a template for hybridizing to a complementary primer (the “second primer”), followed by primer extension to form a second strand that is complementary to the first strand that is formed by ligation of the first and second probes. Extension of the second primer through the 5′ primer-specific portion in the first strand creates a complement of the 5′ primer specific portion, which can serve as a template for hybridizing to a complementary primer (the “first primer”). Primer extension of this first primer can be used to generate a new copy of the first strand.

In some embodiments, the primer-specific portions in each probe set (and thus, the first and second primers that are complementary to the primer-specific portions) are designed to have Tm values that are within a ΔTm range of 10° C. or less, 5° C. or less, 3° C. or less, or 2° C. or less. In some embodiments, the first and second primer-specific portions in a plurality of probe sets are designed to have Tm values that are within a ΔTm range of 10° C. or less, 5° C. or less, 3° C. or less, or 2° C. or less. In some embodiments, the first primer-specific portions in first probes from a plurality of the probe sets are identical to each other, so that a plurality of different ligation products (first strands generated by ligation of first and second probes from different probe sets) can be amplified simultaneously by extending the same first complementary primer. In some embodiments, the second primer-specific portions in second probes from a plurality of the probe sets are identical to each other, so that a plurality of different second strands (generated by forming the complement of the first strand by extending the second primer) can be amplified simultaneously by extending the same second primer. Tm values can be calculated for such primer-specific portions using the references cited above for the target-specific portions.

A “universal primer” is capable of hybridizing to the primer-specific portion of first or second probes from more than one probe set, ligation product, or amplification product, as appropriate. A “universal primer set” comprises a first primer and a second primer that hybridize with a plurality of species of probes, ligation products, or amplification products, as appropriate. In some embodiments, the universal primer or the universal primer set hybridizes with all or most of the probes, ligation products, or amplification products in a reaction, as appropriate. When universal primer sets are used in some amplification reactions, such as, but not limited to, PCR, quantitative results may be obtained for a broad range of template concentrations.

The first or second probe in each set further comprises an identifier tag portion that is between the primer-specific portion and the target-specific portion. The identifier tag portion can be used to identify the probe that contains the identifier tag portion, as explained further below. Thus, the tag sequences should be selected to minimize (1) internal, self-hybridization, (2) hybridization with other same-sequence tags, (3) hybridization with other, different sequence tag complements, (4) and hybridization with the sample polynucleotides. Similar considerations apply to the target-specific portions and the primer-specific portions as well. Also, it is preferred that each identifier tag portion can specifically recognize and hybridize to its corresponding tag portion complement under the same conditions for all tags.

Sequences of identifier tag portions can be selected by any suitable method. For example, computer algorithms for selected non-crosshybridizing sets of tags are described in Brenner (PCT Publications No. WO 96/12014 and WO 96/41011) and Shoemaker (Shoemaker et al., European Pub. No. EP 799897 Al (1997)). Preferably, the tag portions have Tm values that are within a preselected temperature range, as discussed above with respect to the primer-specific portions. Preferably, the melting temperatures of the tag portions are within a ΔTm range of 10° C. or less, 5° C. or less, 3° C. or less, or 2° C. or less. In some embodiments, the tag portions in a plurality or all of the probe sets are designed to have Tm values that are within a ΔTm range of 10° C. or less, 5° C. or less, 3° C. or less, or 2° C or less. Preferably, the tag segments are at least 12 bases in length to facilitate specific hybridization to corresponding tag complements. Typically, tag segments are from 12 to 60 bases in length, and typically from 15 to 30 bases in length.

In another embodiment, the first and second probes of at least one different probe set are provided in a covalently linked form, such that the first probe is covalently linked by its 5′ end to the 3′ end of the second probe by a linking moiety. In one embodiment, the linking moiety comprises a chain of polynucleotides that are not significantly complementary to the target strand, the probes, or to any other nucleic acid in the sample. The linking moiety is sufficiently long to allow the target-complementary sequences in the probes to hybridize to the target strand region and to form a viable hybridization complex for cleavage. Typically, the linking moiety is-longer than, preferably at least 10 nucleotides longer than, the collective length of the first and second target regions. A polynucleotide linking moiety can contain or consist of any suitable sequence. For example, the linking moiety can be a homopolymer of C, T, G or A. Alternatively, the linking moiety can contain or consist of a non-nucleotidic polymer, such as polyethylene glycol, a polypeptide such as polyglycine, etc.

In some embodiments, the primer set further comprises at least one first primer. The first primer of a primer set is designed to hybridize with the complement of the 5′ primer-specific portion of that same ligation or amplification product in a sequence-specific manner. According to some embodiments, a primer set of the present invention comprises at least one second primer. The second primer in that primer set is designed to hybridize with a 3′ primer-specific portion of a ligation or amplification product in a sequence-specific manner. In some embodiments, at least one primer of the primer set comprises a promoter sequence or its complement or a portion of a promoter sequence or its complement. For a discussion of primers comprising promoter sequences, see Sambrook and Russell.

According to some embodiments, some probe sets may comprise more than one first probe or more than one second probe to allow sequence discrimination between target sequences that differ by one or more nucleotides.

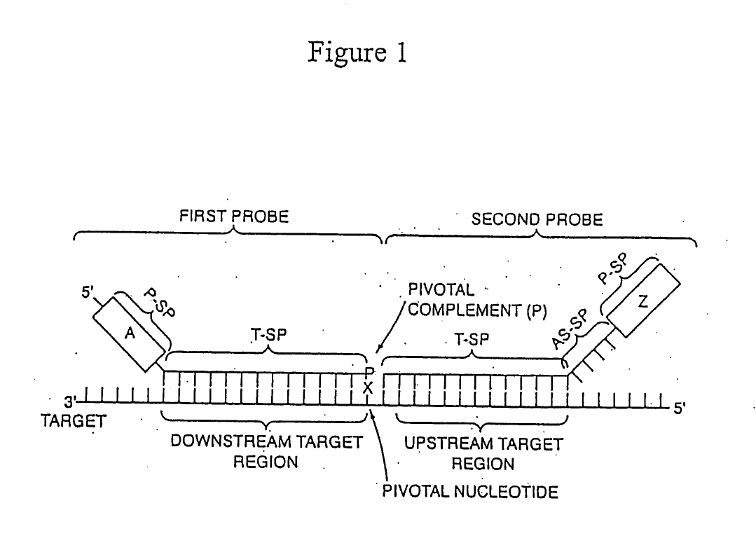

According to some embodiments of the invention, a target-specific probe set can be designed so that the target-specific portion of the first probe will hybridize with the downstream target region (see, e.g., probe A in FIG. 1) and the target-specific portion of the second probe will hybridize with the upstream target region (see, e.g., probe Z in FIG. 1). A nucleotide base complementary to the pivotal nucleotide, the “pivotal complement,” is present on the proximal end of either the first probe (3′ end) or the second probe (5′ end) of the target-specific probe set.

When the first and second probes of the probe set are hybridized to the appropriate upstream and downstream target regions, and the pivotal complement is base-paired with the pivotal nucleotide on the target sequence, the hybridized first and second probes may be ligated together to form a ligation product (see, e.g., FIGS. 1(b)-(c)). A mismatched base at the pivotal nucleotide, however, impedes ligation, even if both probes are otherwise fully hybridized to their respective target regions. Thus, highly related sequences that differ by as little as a single nucleotide can be distinguished.

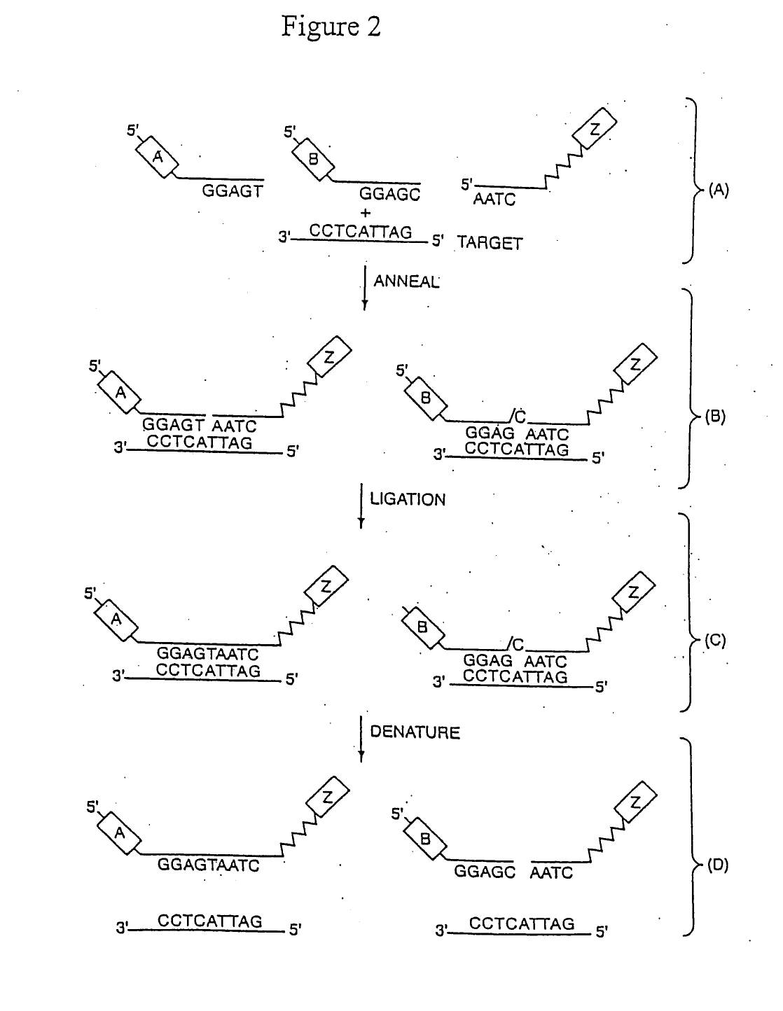

For example, according to some embodiments, one can distinguish the two potential alleles in a biallelic locus as follows. A probe set comprising two first probes, differing in their primer-specific portions and their pivotal complement (see, e.g., probes A and B in FIG. 2(a)) is combined with a second probe (see, e.g., probe Z in FIG. 2(a)) and a sample containing target nucleic acids. All three probes will hybridize with the target sequence under appropriate conditions (see, e.g., FIG. 2(b)). Only the first probe with the hybridized pivotal complement, however, will be ligated with the hybridized second probe (see, e.g., FIG. 2(c)). Thus, if only one allele is present in the sample, only one ligation product for that target will be generated (see, e.g., ligation product A-Z in FIG. 2(d)). Both ligation products would be formed in a sample from a heterozygous individual.

Further, in some embodiments, probe sets do not comprise a pivotal complement at the terminus of the first or the second probe. Rather, the target nucleotide or nucleotides to be detected are located within either the 3′ or 5′ target region to which the first probe or second probe hybridizes. Probes with target-specific portions that are fully complementary with their respective target regions can hybridize under stringent conditions. Probes with one or more mismatched bases in the target-specific portion, by contrast, will not hybridize to their respective target region. Both the first probe and the second probe must be hybridized to the target for a ligation product to be generated. Thus, nucleotides to be detected may be pivotal or internal or both.

In some embodiments, the first probes and second probes in a probe set are designed with similar melting temperatures (Tm). Where a probe includes a pivotal complement, the Tm for the probe(s) comprising the pivotal complement(s) of the target pivotal nucleotide can be designed to be approximately 4-6° C. lower than the Tm values of the other probe(s) that do not contain the pivotal complement in the probe set. The probe comprising the pivotal complement(s) will also preferably be designed with a Tm near the ligation temperature. Thus, in these exemplary embodiments, a probe with a mismatched nucleotide will more readily dissociate from the target at the ligation temperature. Thus, the ligation temperature can provide another way to discriminate between, for example, multiple potential alleles in the target.

A ligation agent according to the present invention may comprise any number of enzymatic or chemical (i.e., non-enzymatic) agents. For example, ligase is an enzymatic ligation agent that, under appropriate conditions, forms phosphodiester bonds between the 3′-OH and the 5′-phosphateof adjacent polynucleotides. Temperature-sensitive ligases, include, but are not limited to, bacteriophage T4 ligase, bacteriophage T7 ligase, and E. coli ligase. Thermostable ligases include, but are not limited to, Taq ligase, Tth ligase, and Pfu ligase. Thermostable ligase may be obtained from thermophilic or hyperthermophilic organisms, including but not limited to, prokaryotic, eucaryotic, or archael organisms. Some RNA ligases may also be employed in the methods of the invention.

Chemical ligation agents include, without limitation, activating, condensing, and reducing agents, such as carbodiimide, cyanogen bromide (BrCN), N-cyanoimidazole, imidazole, 1-methylimidazole/carbodiimide/cystamine, dithiothreitol (DTT) and ultraviolet light. Autoligation, i.e., spontaneous ligation in the absence of a ligating agent, is also within the scope of the invention. Detailed protocols for chemical ligation methods and descriptions of appropriate reactive groups can be found, among other places, in Xu et al., Nucleic Acid Res., 27:875-81 (1999); Gryaznov and Letsinger, Nucleic Acid Res. 21:1403-08 (1993); Gryaznov et al., Nucleic Acid Res. 22:2366-69 (1994); Kanaya and Yanagawa, Biochemistry 25:7423-30 (1986); Luebke and Dervan, Nucleic Acids Res. 20:3005-09 (1992); Sievers and von Kiedrowski, Nature 369:221-24 (1994); Liu and Taylor, Nucleic Acids Res. 26:3300-04 (1999); Wang and Kool, Nucleic Acids Res. 22:2326-33 (1994); Purmal et al., Nucleic Acids Res. 20:3713-19 (1992); Ashley and Kushlan, Biochemistry 30:2927-33 (1991); Chu and Orgel, Nucleic Acids Res. 16:3671-91 (1988); Sokolova et al., FEBS Letters 232:153-55 (1988); Naylor and Gilham, Biochemistry 5:2722-28 (1966); U.S. Pat. No. 5,476,930; and Royer, EP 324616B1). In some embodiments, the ligation agent is an “activating” or reducing agent. It will be appreciated that if chemical ligation is used, the 3′ end of the first probe and the 5′ end of the second probe should include appropriate reactive groups to facilitate the ligation.

In some embodiments, for amplification, a polymerase is used. In some embodiments, the polymerase may comprise at least one thermostable polymerase, including, but not limited to, Taq, Pfu, Vent, Deep Vent, Pwo, UITma, and Tth polymerase and enzymatically active mutants and variants thereof. Such polymerases are well known and/or are commercially available. Descriptions of polymerases can be found, among other places, at the world wide web URL: the-scientist.library.upenn.edu/yr1998/jan/profile 1_{—}980105. html.

The invention also employs probes that are useful for detecting amplified ligation products using a mobility- or mass-dependent analysis technique. Each mobility probe comprises (a) a mobility defining moiety that imparts an identifying mobility or total mass to the mobility probe, and (b) a tag portion or tag portion complement for hybridizing to a complementary tag portion complement or tag portion, respectively, in an amplified strand. For each different target sequence to be detected (e.g., for a different locus or for a particular SNP), a different mobility probe is prepared which has a distinct tag portion or tag portion complement, and a distinct mobility defining moiety which allows the attached tag portion or tag portion complement (and the corresponding target sequence) to be identified from the distinct mobility or total mass of the mobility probe.

Any of a variety of different probe constructs and configurations can be used. In the following discussion, although the mobility defining moiety is referred to as a “tail” or “tail portion”, such wording is not indended to limit the structure of the mobility defining moiety.

The tail portion of a mobility defining moiety may be any entity capable of achieving a particular mobility or total mass. In certain embodiments, the tail portion of the mobility defining moiety of the invention should (1) have a low polydispersity in order to effect a well-defined and easily resolved mobility, e.g., Mw/Mn less than 1.05; (2) be soluble in an aqueous medium; (3) not adversely affect probe-target hybridization; and (4) be available in sufficient number such that mobility probes for different probe sets have distinguishable mobilities or total masses.

In certain embodiments, the tail portion comprises a polymer. For example, the polymer may be homopolymer, random copolymer, or block copolymer. Furthermore, the polymer may have a linear, comb, branched, or dendritic structure. In addition, although the invention is described herein with respect to a single polymer chain attached to an associated mobility defining moiety, the invention also contemplates mobility defining moieties comprising more than one polymer chain element, where the elements collectively form a tail portion.

Exemplary polymers for use in the present invention include, but are not limited to, hydrophilic, or at least sufficiently hydrophilic when bound to a tag complement to ensure that the tag complement is readily soluble in aqueous medium. Where the mobility-dependent analysis technique is electrophoresis, the polymers can be designed for some embodiments of the invention to be uncharged or have a charge/subunit density that is substantially less than that of the amplification product.

In certain embodiments, the polymer comprises polyethylene oxide (PEO), e.g., formed from one or more hexaethylene oxide (HEO) units, where the HEO units are joined end-to-end to form an unbroken chain of ethylene oxide subunits. Other exemplary embodiments include a chain composed of n 12mer PEO units, and a chain composed of n tetrapeptide units, where n is an adjustable integer (e.g., Grossman et al., U.S. Pat. No. 5,777,096).

In certain embodiments, the synthesis of polymers useful as tail portions may depend on the nature of the polymer. Methods for preparing suitable polymers generally follow well known polymer subunit synthesis methods. Methods of forming selected-length PEO chains are discussed below. These methods, which involve coupling of defined-size, multi-subunit polymer units to one another, either directly or through charged or uncharged linking groups, are generally applicable to a wide variety of polymers, such as polyethylene oxide, polyglycolic acid, polylactic acid, polyurethane polymers, polypeptides, and oligosaccharides. Such methods of polymer unit coupling are also suitable for synthesizing selected-length copolymers, e.g., copolymers of polyethylene oxide units alternating with polypropylene units. Polypeptides of selected lengths and amino acid composition, either homopolymer or mixed polymer, can be synthesized by standard solid-phase methods (e.g., Fields and Noble, Int. J. Peptide Protein Res., 35: 161-214 (1990)).

In some methods for preparing PEO polymer chains having a selected number of HEO units, an HEO unit is protected at one end with dimethoxytrityl (DMT), and activated at its other end with methane sulfonate. The activated HEO is then reacted with a second DMT-protected HEO group to form a DMT-protected HEO dimer. This unit-addition is then carried out successively until a desired PEO chain length is achieved (e.g., Levenson et al., U.S. Pat. No. 4,914,210).

Another exemplary polymer for use as a tag portion complement is L-DNA. L-DNA polymers can be prepared by standard oligonucleotide synthesis as described above, form the corresponding L-DNA monomers, which are commercially available. One advantage of L-DNA polymers is that they do not hybridize to standard D-DNA polymers, so cross-hybridization problems are reduced.

Coupling of the polymer tails to a polynucleotide tag complement can be carried out by an extension of conventional phosphoramidite polynucleotide synthesis methods, or by other standard coupling methods, e.g., a bis-urethane tolyl-linked polymer chain may be linked to a polynucleotide on a solid support via a phosphoramidite coupling. Alternatively, the polymer chain can be built up on a polynucleotide (or other tag portion) by stepwise addition of polymer-chain units to the polynucleotide, e.g., using standard solid-phase polymer synthesis methods.

The contribution of the tail to the mobility of the probe in some embodiments, will generally depend on the size of the tail. However, addition of charged groups to the tail, e.g., charged linking groups in the PEO chain, or charged amino acids in a polypeptide chain, can also be used to achieve selected mobility or mass characteristics.

Additional guidance for selection and synthesis of mobility defining moieties can be found in PCT Publications No. WO 00/55368 (Grossman), WO 01/49790 (Menchen et al.), and WO 02/83954 (Woo et al, application No. PCT/US02/11824).

When a tag portion or tag portion complement is a polynucleotide, the tag complement may comprise all, part, or none of the tail portion of the mobility defining moiety. In some embodiments of the invention, the tag portion or tag portion complement may consist of some or all of the tail portion. In other embodiments of the invention, the tag portion or tag portion complement does not comprise any portion of the tail portion of the mobility defining moiety. For example, because PNA is uncharged, particularly when using free solution electrophoresis as the mobility-dependent analysis technique, the same PNA oligomer may act as both a tag portion complement and a tail portion of a mobility defining moiety. One advantage of including PNA in the tag portion complement is that it is uncharged, so that the mobility of the mobility probe is reduced relative to the same probe containing DNA instead of PNA.

In some embodiments, the mobility probe may include a hybridization enhancer, where, as used herein, the term “hybridization enhancer” means moieties that serve to enhance, stabilize, or otherwise positively influence hybridization between two polynucleotides, e.g. intercalators (e.g., U.S. Pat. No. 4,835,263), minor-groove binders (e.g., U.S. Pat. No. 5,801,155), and cross-linking functional groups. In some embodiments, the hybridization enhancer is covalently attached to the mobility defining moiety. In some embodiments, a hybridization enhancer for use in the present invention is a minor-groove binder, e.g., netropsin, distamycin, or the like.

In some embodiments, the mobility probes may include a detectable label to facilitate detection of the mobility probe, such as a fluorescent moiety. The skilled artisan will appreciate that many such labels are known in the art, such as fluorophores, radioisotopes, chromogens, enzymes, antigens, heavy metals, dyes, magnetic probes, phosphorescence groups, chemiluminescent groups, and electrochemical detection moieties. Exemplary fluorophores include, but are not limited to, rhodamine, cyanine 3 (Cy 3), cyanine 5 (Cy 5), fluorescein, Vic™, Liz™, Tamra™, 5-Fam™, 6-Fam™, and Texas Red (Molecular Probes). (Vic™, Liz™, Tamra™, 5-Fam™, and 6-Fam™ are all available from Applied Biosystems, Foster City, Calif.) Exemplary radioisotopes include, but are not limited to, ³²P, ³³P, and ³⁵S. Reporter groups also include elements of multi-element indirect reporter systems, e.g., biotin/avidin, antibody/antigen, ligand/receptor, enzyme/substrate, and the like, in which the element interacts with other elements of the system in order to effect a detectable signal. One exemplary multi-element reporter system includes a biotin reporter group attached to a primer and an avidin conjugated with a fluorescent label. Detailed protocols for methods of attaching detectable labels to oligonucleotides and polynucleotides can be found in, among other places, G. T. Hermanson, Bioconjugate Techniques, Academic Press, San Diego, Calif. (1996) and S. L. Beaucage et al., Current Protocols in Nucleic Acid Chemistry, John Wiley & Sons, New York, N.Y. (2000).

In some embodiments, the label comprises a fluorescent moiety (also called a “fluorescent dye”) that comprises a resonance-delocalized system or aromatic ring system that absorbs light at a first wavelength and emits fluorescent light at a second wavelength in response to the absorption event. A wide variety of such dye molecules are known in the art. For example, fluorescent dyes can be selected from any of a variety of classes of fluorescent compounds, such as xanthenes, rhodamines, fluoresceins, cyanines, phthalocyanines, squaraines, and bodipy dyes.

In one embodiment, the dye comprises a xanthene-type dye, which contains a fused three-ring system of the form:

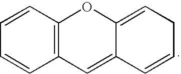

This parent xanthene ring may be unsubstituted (i.e., all substituents are H) or may be substituted with one or more of a variety of the same or different substituents, such as described below.

In one embodiment, the dye contains a parent xanthene ring having the general structure:

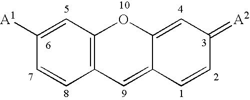

In the parent xanthene ring depicted above, A¹ is OH or NH₂ and A² is O or NH₂+. When A¹ is OH and A² is O, the parent xanthene ring is a fluorescein-type xanthene ring. When A¹is NH₂ and A² is NH₂+, the parent xanthene ring is a rhodwnine-type xanthene ring. When A¹ is NH₂ and A² is O, the parent xanthene ring is a rhodol- type xanthene ring. In the parent xanthene ring depicted above, one or both nitrogens of A¹ and A² (when present) and/or one or more of the carbon atoms at positions C1, C2, C4, C5, C7, C8 and C9 can be independently substituted with a wide variety of the same or different substituents. In one embodiment, typical substituents include, but are not limited to, —X, —R, —OR, —SR, —NRR, perhalo (C₁-C₆) alkyl, —CX₃, —CF₃, —CN, —OCN, —SCN, —NCO, —NCS, —NO, —NO₂, —N₃, —S(O)₂O⁻, —S(O)₂OH, —S(O)₂R, —C(O)R, —C(O)X, —C(S)R, —C(S)X, —C(O)OR, —C(O)O⁻, —C(S)OR, —C(O)SR, —C(S)SR, —C(O)NRR, —C(S)NRR and —C(NR)NRR, where each X is independently a halogen (preferably —F or Cl) and each R is independently hydrogen, (C₁-C₆) alkyl, (C₁-C₆) alkanyl, (C₁-C₆) alkenyl, (C₁-C₆) alkynyl, (C₅-C₂₀) aryl, (C₆-C₂₆) arylalkyl, (C₅-C₂₀) arylaryl, heteroaryl, 6-26 membered heteroarylalkyl 5-20 membered heteroaryl-heteroaryl, carboxyl, acetyl, sulfonyl, sulfinyl, sulfone, phosphate, or phosphonate. Moreover, the C1 and C2 substituents and/or the C7 and C8 substituents can be taken together to form substituted or unsubstituted buta[1,3]dieno or (C₅-C₂₀) aryleno bridges. Generally, substituents which do not tend to quench the fluorescence of the parent xanthene ring are preferred, but in some embodiments quenching substituents may be desirable. Substituents that tend to quench fluorescence of parent xanthene rings are electron-withdrawing groups, such as —NO₂, —Br, and —I. In one embodiment, C9 is unsubstituted. In another embodiment, C9 is substituted with a phenyl group. In another embodiment, C9 is substituted with a substituent other than phenyl.

When A¹ is NH₂ and/or A² is NH₂+, these nitrogens can be included in one or more bridges involving the same nitrogen atom or adjacent carbon atoms, e.g., (C₁-C₁₂) alkyldiyl, (C₁-C₁₂) alkyleno, 2-12 membered heteroalkyldiyl and/or 2-12 membered heteroalkyleno bridges.

Any of the substituents on carbons C1, C2, C4, C5, C7, C8, C9 and/or nitrogen atoms at C3 and/or C6 (when present) can be further substituted with one or more of the same or different substituents, which are typically selected from —X, —R′, ═O, —OR′, —SR′, ═S, —NR′R′, ═NR′, —CX₃, —CN, —OCN, —SCN, —NCO, —NCS, —NO, —NO₂, ═N₂, —N₃, —NHOH, —S(O)₂O⁻, —S(O)₂OH, —S(O)₂R′, —P(O)(O⁻)₂, —P(O)(OH)₂, —C(O)R′, —C(O)X, —C(S)R′, —C(S)X, —C(O)OR′, —C(O)O⁻, —C(S)OR′, —C(O)SR′, —C(S)SR′, —C(O)NR′R′, —C(S)NR′R′ and —C(NR)NR′R′, where each X is independently a halogen (preferably —F or —Cl) and each R′ is independently hydrogen, (C₁-C₆) alkyl, 2-6 membered heteroalkyl, (C₅-C₁₄) aryl or heteroaryl, carboxyl, acetyl, sulfonyl, sulfinyl, sulfone, phosphate, or phosphonate.

Exemplary parent xanthene rings include, but are not limited to, rhodamine-type parent xanthene rings and fluorescein-type parent xanthene rings.

In one embodiment, the dye contains a rhodamine-type xanthene dye that includes the following ring system:

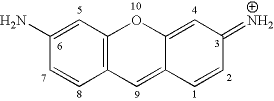

In the rhodamine-type xanthene ring depicted above, one or both nitrogens and/or one or more of the carbons at positions C1, C2, C4, C5, C7 or C8 can be independently substituted with a wide variety of the same or different substituents, as described above for the parent xanthene rings, for example. C9 may be substituted with hydrogen or other substituent, such as an orthocarboxyphenyl or ortho(sulfonic acid)phenyl group. Exemplary rhodamine-type xanthene dyes include, but are not limited to, the xanthene rings of the rhodamine dyes described in U.S. Pat. Nos. 5,936,087, 5,750,409, 5,366,860, 5,231,191, 5,840,999, 5,847,162,and 6,080,852 (Lee et al.), PCT Publications WO 97/36960 and WO 99/27020, Sauer et al., J. Fluorescence 5(3):247-261 (1995), Arden-Jacob, Neue Lanwellige Xanthen-Farbstoffe für Fluoreszenzsonden und Farbstoff Laser, Verlag Shaker, Germany (1993), and Lee et al., Nucl. Acids Res. 20:2471-2483 (1992). Also included within the definition of “rhodamine-type xanthene ring” are the extended-conjugation xanthene rings of the extended rhodamine dyes described in U.S. Pat. No. No. 6,248,884.

In another embodiment, the dye comprises a fluorescein-type parent xanthene ring having the structure:

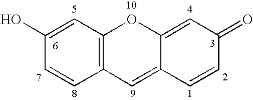

In the fluorescein-type parent xanthene ring depicted above, one or more of the carbons at positions C1, C2, C4, C5, C7, C8 and C9 can be independently substituted with a wide variety of the same or different substituents, as described above for the parent xanthene rings. C9 may be substituted with hydrogen or other substituent, such as an orthocarboxyphenyl or ortho(sulfonic acid)phenyl group. Exemplary fluorescein-type parent xanthene rings include, but are not limited to, the xanthene rings of the fluorescein dyes described in U.S. Pat. Nos. 4,439,356, 4,481,136, 4,933,471 (Lee), U.S. Pat. No. 5,066,580 (Lee), U.S. Pat. Nos. 5,188,934, 5,654,442, and 5,840,999, WO 99/16832, and EP 050684. Also included within the definition of “fluorescein-type parent xanthene ring” are the extended xanthene rings of the fluorescein dyes described in U.S. Pat. Nos. 5,750,409 and 5,066,580.

In another embodiment, the dye comprises a rhodamine dye, which comprises a rhodamine-type xanthene ring in which the C9 carbon atom is substituted with an orthocarboxy phenyl substituent (pendent phenyl group). Such compounds are also referred to herein as orthocarboxyfluoresceins. A particularly preferred subset of rhodamine dyes are 4,7,-dichlororhodamines. Typical rhodamine dyes include, but are not limited to, rhodamine B, 5-carboxyrhodamine, rhodamine X (ROX), 4,7-dichlororhodamine X (dROX), rhodamine 6G (R6G), 4,7-dichlororhodamine 6G, rhodamine 110 (RI 10), 4,7-dichlororhodamine 110 (dR110), tetramethyl rhodamine (TAMRA) and 4,7-dichloro-tetramethylrhodamine (dTAMRA). Additional rhodamine dyes can be found, for example, in U.S. Pat. No. 5,366,860 (Bergot et al.), U.S. Pat. No. 5,847,162 (Lee et al.), U.S. Pat. No. 6,017,712 (Lee et al.), U.S. Pat. No. 6,025,505 (Lee et al.), U.S. Pat. No. 6,080,852 (Lee et al.), U.S. Pat. No. 5,936,087 (Benson et al.), U.S. Pat. No. 6,111,116 (Benson et al.), U.S. Pat. No. 6,051,719 (Benson et al.), U.S. Pat. Nos. 5,750,409, 5,366,860, 5,231,191, 5,840,999, and 5,847,162, U.S. Pat. No. 6,248,884 (Lam et al.), PCT Publications WO 97/36960 and WO 99/27020, Sauer et al., 1995, J. Fluorescence 5(3):247-261, Arden-Jacob, Neue Lanwellige Xanthen-Farbstoffe für Fluoresenzsonden und Farbstoff Laser, Verlag Shaker, Germany (1993), and Lee et al., Nucl. Acids Res. 20(10):2471-2483 (1992), Lee et al., Nucl. Acids Res. 25:2816-2822 (1997), and Rosenblum et al., Nucl. Acids Res. 25:4500-4504 (1997), for example. In one embodiment, the dye comprises a 4,7-dichloro-orthocarboxyrhodamine.

In another embodiment, the dye comprises a fluorescein dye, which comprises a fluorescein-type xanthene ring in which the C9 carbon atom is substituted with an orthocarboxy phenyl substituent (pendent phenyl group). A preferred subset of fluorescein-type dyes are 4,7,-dichlorofluoresceins. Typical fluorescein dyes include, but are not limited to, 5-carboxyfluorescein (5-FAM), 6-carboxyfluorescein (6-FAM). Additional typical fluorescein dyes can be found, for example, in U.S. Pat. Nos. 5,750,409, 5,066,580, 4,439,356, 4,481,136, 4,933,471 (Lee), U.S. Pat. No. 5,066,580 (Lee), U.S. Pat. No. 5,188,934 (Menchen et al.), U.S. Pat. No. 5,654,442 (Menchen et al.), U.S. Pat. No. 6,008,379 (Benson et al.), and U.S. Pat. No. 5,840,999, PCT publication WO 99/16832, and EPO Publication 050684. In one embodiment, the dye comprises a 4,7-dichloro-orthocarboxyfluorescein.

In other embodiments, the dye can be a cyanine, phthalocyanine, squaraine, or bodipy dye, such as described in the following references and references cited therein: U.S. Pat. No. 5,863,727 (Lee et al.), U.S. Pat. No. 5,800,996 (Lee et al.), U.S. Pat. No. 5,945,526 (Lee et al.), U.S. Pat. No. 6,080,868 (Lee et al.), U.S. Pat. No. 5,436,134 (Haugland et al.), U.S. Pat. No. 5,863,753 (Haugland et al.), U.S. Pat. No. 6,005,113 (Wu et al.), and WO 96/04405 (Glazer et al.).

Exemplary Methods

The present invention, in some embodiments, provides a method for detecting at least one target sequence in a sample. In the method, a sample that contains, or may contain, a plurality of target sequences is combined with a plurality of different probe sets. Each probe set comprises (a) a first probe comprising a first target-specific portion and a 5′ primer-specific portion, and (b) a second probe comprising a second target-specific portion and a 3′ primer-specific portion, wherein the first and second probes in each set are suitable for ligation together when hybridized to adjacent complementary target sequences. The first or second probe in each set further comprises an identifier tag portion that is between the primer-specific portion and the target-specific portion. The identifier tag portion identifies the probe that contains the identifier tag portion.

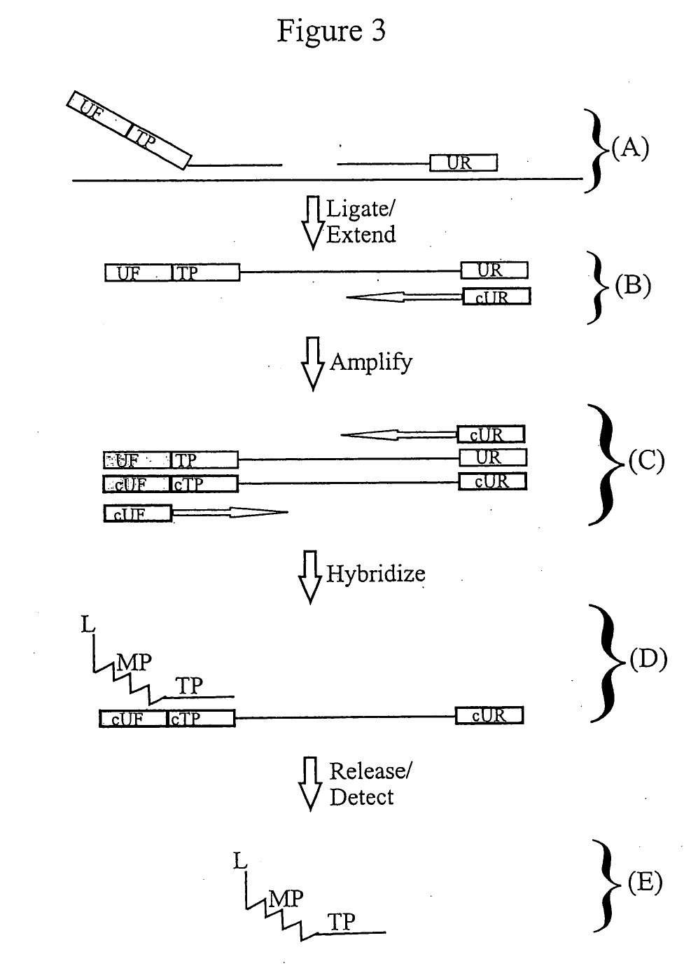

Various exemplary embodiments will now described with reference to FIGS. 3 through 5, which are provided solely for purposes of illustration and not to limit the invention.

In FIG. 3, a target sequence is treated with a plurality of different probe sets for the purposes of detecting a plurality of target nucleic acid sequences. FIG. 3(A) shows a probe set comprises a first probe and a second probe. The first probe comprises a first target-specific portion, an upstream tag portion, and a further upstream 5′ universal forward primer-specific portion. The second probe comprises a second target-specific portion and a downstream universal reverse primer-specific portion. For this example, the first probes in all probe sets contain the same universal 5′ “forward” primer-specific portion, and the second probes in all probe sets contain the same universal 3′ “reverse” primer-specific portion. Following hybridization, such probes can form a complex that is suitable for ligation. After ligation, the resulting ligation product comprises a 5′ primer-specific portion (UF), first and second target-specific portions, a 3′ primer-specific portion (UR), and an identifier tag portion (TP).

In some embodiments, the first and second probes are separated by a gap of one or two nucleotides when the probes are bound to adjacent (nearly adjacent) complementary target sequences. Thus, in some embodiments, the invention also encompasses ligation techniques such as gap-filling ligation, including, without limitation, gap-filling OLA and gap-filling LCR, bridging oligonucleotide ligation, and correction ligation. Descriptions of these techniques can be found, among other places, in U.S. Pat. No. 5,185,243, published European Patent Applications EP 320308 and EP 439182, and published PCT Patent Application WO 90/01069. As discussed above, chemical ligation may also be used, with or without a gap between the adjacent ends of the first and second probes.

Following ligation, FIG. 3(B) shows a complementary universal reverse primer hybridized to the ligation product, for forming the complement of the ligation product by primer extension. Such extension allows subsequent exponential amplification of the ligation product when both universal primers are present, thereby forming a double stranded product comprising a first strand and a second strand that is hybridized to the first strand (FIG. 3(C)).

The amplification product can then be treated with a mobility probe comprising a mobility-defining moiety, a tag portion, and a label (FIG. 3(D)). The tag portion, which imparts an identifying mobility or total mass to the mobility probe, is hybridized to the tag portion complement in the amplified product. The hybridized (bound) mobility probe can be released and subsequently detected by virtue of the mobility probe's label, thereby identifying the target sequence (FIG. 3(E)).

It will be recognized that variations of the scheme presented in FIG. 3 can be implemented. For example, unincorporated probes and primers can be removed at any of variety of stages, using various experimental techniques. In one embodiment, using an affinity capture technique, streptavidin-based capture of biotinylated probes can be performed prior to or following the ligation reaction. It will be appreciated that the upstream (first) probe, the downstream (second) probe, or both, can be biotinylated. In a further embodiment, streptavidin-based capture of biotinylated reverse probe can be performed prior to or after the ligation reaction. A further streptavidin-based capture can be performed following formation of the mobility probe complexes, thereby removing undesired reaction components.

In some embodiments, undesired or unreacted reaction components can be removed by size exclusion chromatography. For example, purification can be performed using Microcon-100 columns, which are commercially available from Millipore, Medford, Mass., by following the manufacturer's instructions.

In other embodiments, an exonuclease that is specific for unhybrized polynucleotides that have a 5′ phosphate group (or 3′ hydroxyl) can be used to selectively degrade unwanted residual unligated probes and/or primers (e.g., see Barany et al., U.S. Pat. No. 6,268,148).

It will be recognized that a variety of mobility-dependent analysis techniques (MDAT) may be employed for the purposes of measuring the mobility probe, such techniques including, but not limited to, electrophoresis, such as gel or capillary electrophoresis, HPLC, mass spectroscopy, including MALDI-TOF, gel filtration and chromatography.

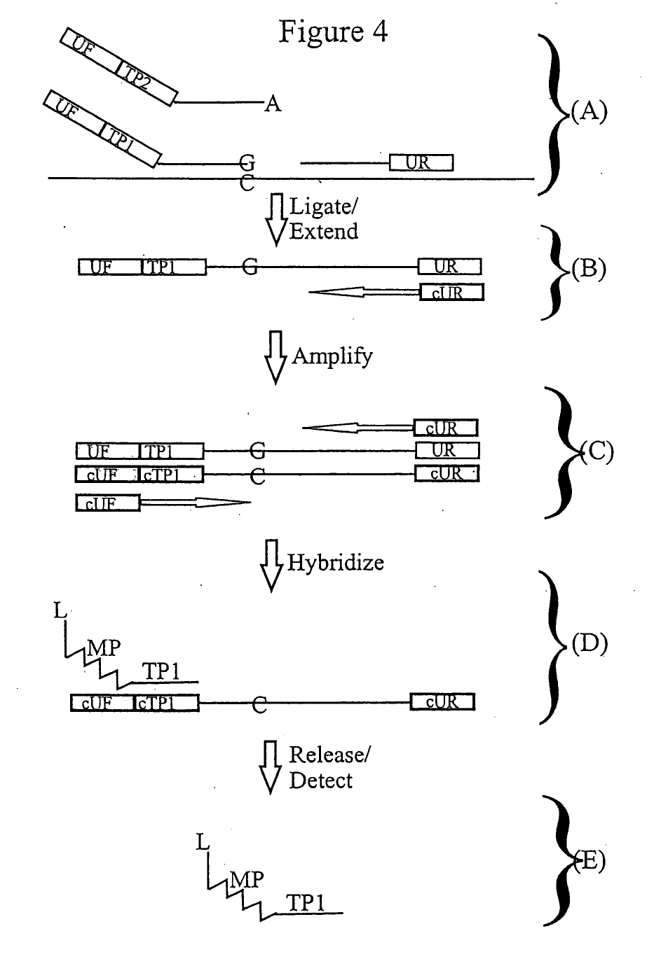

FIG. 4 illustrates how the procedure from FIG. 3 may be used to detect single nucleotide polymorphism (SNP) allelic variants. In FIG. 4(A), the probe set comprises a first probe and a second probe. The first probe comprises a first target-specific portion, an upstream tag portion, an upstream 5′ universal forward primer-specific portion, and a polymorphic G nucleotide. In addition, the probe set also comprises a different first probe comprising a first target-specific portion, an upstream tag portion, a distal 5′ universal forward primer-specific portion, and a polymorphic A nucleotide. The second probe comprises a second target-specific portion and a downstream universal reverse primer-specific portion.

As shown, following hybridization, the complementary first probe that contains the G allele forms a ligation-competent complex with the target stand, but the A allele does not. Thereafter, the steps discussed in FIG. 3 are employed, resulting in detection of the target allele, which is homozygous in this example.

It will be recognized that the invention is not limited solely to genomic SNPs, but may also be practiced in the context of mRNA splice variant detection. In such embodiments, different mRNAs formed by alternative splicing (splice variants) can be reverse-transcribed into cDNA using methods well known in the art.

In addition to detection of sequence variants, some embodiments can be practiced to measure the relative expression levels of different mRNAs. In another embodiment, this may be accomplished by incorporating a promoter sequence, such as the promoter sequence for the T7 RNA polymerase. By incorporation into a ligation or amplification product, multiple rounds of T7 polymerase-mediated linear amplification can be performed, and the resulting amplification products can be detected and/or measured via hybridization, release, and detection of mobility probes.

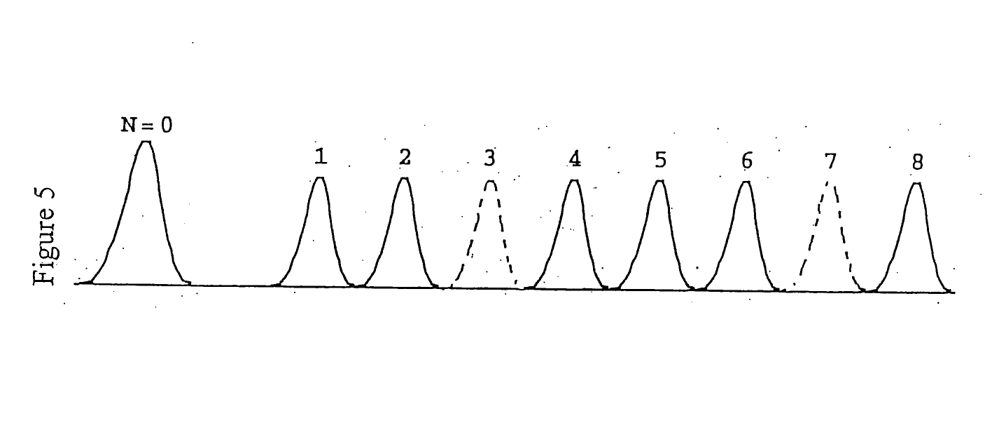

FIG. 5 shows an exemplary schematic electropherogram obtained by electrophoresis of a 8 different mobility probes. Observed peaks are shown by solid lines, and expected but absent peaks are shown by dashed lines. As can be seen, mobility probes number 1, 2, 4, 5, 6, and 8 are observed, indicating the presence of the corresponding target sequences in the sample. Peaks for mobility probes 3 and 7 are absent, indicating that the corresponding target sequences are absent from the sample or are present at levels too small to be detected.

The invention is further illustrated by way of the following example which is not intended to limit the invention in any way.

### EXAMPLE

A probe set is prepared for each target nucleic acid sequence, each set comprising first and second ligation probes designed to hybridize adjacently to the desired complementary target sequences. For a 5-plex assay, ten probes sets can be prepared for detecting five pairs of alternate alleles at five different loci. The ten probe sets include five pairs of locus-specific probe sets, wherein each pair is designed to detect two possible alternative single nucleotide polymorphisms (SNPs) in a particular locus.

The first probe in each set comprises a 5′ primer-specific portion at its 5′ end, a first target-specific portion at its 3′ end, and an identifier tag portion between the primer-specific portion and the target-specific portion. For this example, the sequence of the 5′ primer-specific portion of each of the one or more first probes is identical, and, for this example, has a length of 18-22 nucleotides and a Tm of 55-65° C.

The target specific portion of each first probe comprises a sequence that is complementary to a different target sequence in the sample. In this example, each pair of probe sets comprises two different first probes which contain identical target-specific portions except for the presence of a different 3′terminal nucleotide, for hybridizing to either of two alternative SNPs at the same locus. The target-specific portions may be designed to have approximately the same Tm values, all within approximately 2 or 3° C. at 10 nM. Exemplary ranges are as follows: 42-44° C., 53-55° C., or 57-60° C. In some embodiments, target-specific portions are designed to be approximately 17-25 nucleotides in length.

The identifier tag portion in each first probe comprises a distinct sequence that can be used to identify the particular target-specific portion in the probe. In this example, the identifier tag portions are designed to have approximately the same Tm (65-68° C.) and are from 22-26 nucleotides in length.

The second probe in each set comprises a target-specific portion at its 5′ end and a 3′ primer-specific portion at its 3′ end. In this example, the first and second probes in each set are designed to hybridize to their complementary target sequences, such that the 3′ end of the first probe abuts the 5′ end of the second probe. The resulting “nick complex” can be ligated.

For this example, the sequence of the 3′ primer-specific portion of the second probes in each probe set is identical, and, for this example, has a length of 18-22 nucleotides and a Tm of 55-65° C. In some embodiments, the Tm of the 3′ primer-specific portion is about 3° C. higher than the Tm of the 5′ primer-specific portion.

The probe sets are combined with a genomic DNA sample from human blood (Coriell Institute for Medical Research, Camden, N.J.) to form a ligation reaction mixture (10 μL) comprising sample gDNA (10 ng/μL), 20 mM Tris-HCl, pH 7.6, 25 mM potassium acetate, 10 mM magnesium acetate, 10 mM DTT, 1 mM NAD, 0.1% Triton X-100, 10 nM of each first probe, 20 nM of each second probe, and 3 to 10 U ligase (from 0.12 to 1.0 U/μL) (Taq ligase mutant AK16D, Nucl. Acids Res. 27:788 (1999), or Taq ligase (New England BioLabs, Beverly, Mass.).

The ligation reaction mixture is pre-heated with a 9700 Thermocycler (Applied Biosystems, Foster City, Calif.) at 95° C. for 2 minutes, followed by 80° C. for 1 minute during which the ligase is added. Ligation products may be generated using thermocycling conditions of: 10-40 cycles at 90° C. for 10 seconds and 55-60° C. for 4 minutes. After the cycling, the mixture is optionally heated at 95° C. for 10-20 minutes. In another exemplary protocol, the reaction mixture is pre-heated at 90° C. for 3 minutes, followed by 10-40 thermocycles (90° C. for 15 seconds and 55° C. for 5 minutes), followed by heating at 95° C. for 10-20 minutes (optional) and a 4° C. hold.

Following ligation, streptavidin magnetic (SAV-Mag) beads can be used to select biotinylated ligation products. For example, the second probe in each probe set includes a 3′ biotin moiety for streptavidin capture. For example, 10 μL of SAV-Mag beads (10⁶-10⁷ beads/μL, 0.7 μm diameter, Seradyn, Indianapolis, Ind.) are added to the 10 μL ligation reaction mixture and incubated at 25° C. or ambient temperature for 10-30 minutes. After incubation, a magnet is placed at the bottom of the sample for 2 minutes, and the supernatant is removed by micropipette. The beads are then washed in 100 μL 1× phosphate buffered saline containing 0.1% Tween-20. After the wash, the magnet is then placed near the bottom of the sample for 2 minutes, and the supernatant is removed by micropipette.

Amplification can be performed by PCR by suspending the bead-immobilized ligation products in an amplification solution (10 μL) comprising 5 μL of Amplitaq Gold PCR Master Mix (Applied Biosystems)+5 μL water, and 1 μM each (final concentration) of first and second universal primers (the first primer is complementary to the complement of the 5′ primer-specific portions of each first probe, and the second primer is complementary to the 3′ primer-specific portions of each second probe).

Alternatively, if the sample was not purified using streptavidin bead capture, an amplification mixture can be prepared by transferring an aliquot of the ligation reaction mixture (1 μL) to an amplification solution (9 μL) to produce final concentrations as just described.

The amplification reaction mixture is pre-heated at 95° C. for 10 minutes, followed by 25-30 cycles using 92° C. for 15 seconds, 55° C. for 60 seconds, 72° C. for 30 seconds, ending with 72° C. for 7 minutes and 4° C. hold.

In some embodiments, a post-amplification purification is performed by adding 10 μL of SAV-Mag beads (10⁷ beads/uL, 0.7 μm diameter, Seradyn) to the 10 μL amplification reaction mixture and incubated at ambient temperature for 10-30 minutes. Next, 10 μL of 0.1M NaOH is added and the resulting mixture is incubated at ambient temperature for 10-20 minutes. After the incubation, a magnet is placed near the bottom of the mixture for 0.5-2 minutes, and the supernatant is removed by micropipette.

For detection of the different amplified ligation products, mobility probes can be prepared for hybridization to the tag portions or tag portion complements of the amplified strands, such that each mobility probe can be used to identify a particular target sequence for which the corresponding probe set was successfully ligated and amplified. For example, each mobility probe can comprise a tag portion or tag portion complement comprising a polynucleotide sequence (e.g., 22-26 nt) that is specific for the corresponding tag portion or tag portion complement in one of the amplified strands. Each mobility probe additionally comprises a mobility defining moiety that imparts an identifying mobility (e.g., for electrophoretic detection) or total mass (for detection by mass spectrometry) to the mobility probe. For example, the mobility probe for each different target sequence may comprise a polyethylene glycol (PEO) polymer segment having a different length (EO)n, where n ranges from 1 to 10. For fluorescence detection, the mobility probes may additionally include fluorescent dyes, such as FAM and VIC dyes, for detection of the different, alternative SNPs at each target locus. These may be attached by standard linking chemistries to the “5′ end” of the mobility defining moiety (the end of the mobility defining moiety that is opposite to end that is linked to the tag portion or tag portion complement).

The mobility probes may be hybridized to amplified strands as follow. To the bead-immobilized amplification products is added 10 μL of a mixture of mobility probes (final concentration 100 pM to 1 nM each, in 4×SSC buffer containing 0.1% SDS), and the resulting mixture is incubated at 50° C. 60° C. for 30 minutes. After the incubation, 100-200 μL 1× PBS buffer containing 0.1% Tween-20 is added. After the mixture is vortexed, a magnet is placed near the bottom of the mixture tube for 2 minutes, and the supernatant is removed by micropipette, and this process of adding PBS buffer, vortexing, and removing supernatant is repeated twice more. A final wash is performed with 0.1× PBS containing 0.1% Tween-20, followed by vortexing and removal of supernatant. To the beads are added 10 μL of DI-formamide solution (Applied Biosystems) and 0.25 μL of size standards (LIZ 120™, Applied Biosystems). The resulting mixture is heated to 95° C. for 5 minutes, and an aliquot is loaded by electrokinetic injection (30 sec at 1.5 kV) onto a 36 cm long capillary tube loaded with POP6™ (Applied Biosystems) on an ABI Prism 3100 Genetic Analyzer™, 15 kV run voltage, 60° C. for 20 minutes using a FAM and VIC Matrix.

In the resulting electropherogram, fluorescent peaks are observed for different mobility probes, due to their distinct combinations of mobility and fluorescent label. The mobility and fluorescent signal for each mobility probe is usually already known from prior experimentation, so that the corresponding target sequences can be readily identified. In some embodiments, two different mobility probes may migrate with the same mobility, but they can be distinguished if they comprise different labels (e.g., FAM and VIC). In other embodiments, each mobility probe is designed to migrate with a distinct mobility, and the attached fluorescent label alternates between FAM and VIC for each successive peak, to further simplify identification of the probes. A size standard can also be used to facilitate identification of the probes.

System

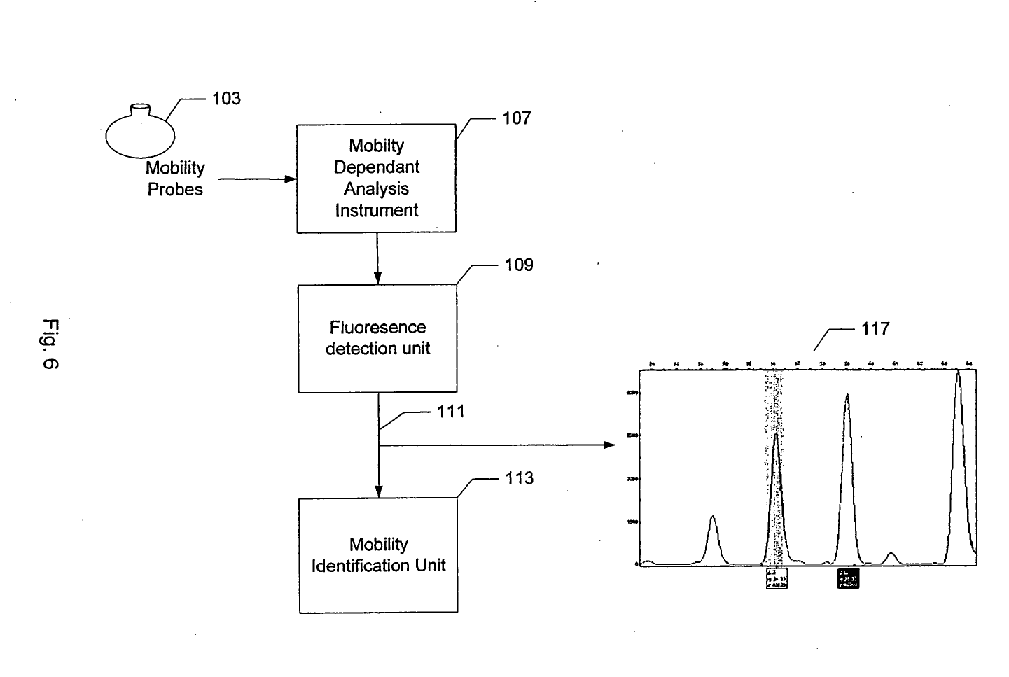

In various embodiments of a system, in accordance with the teachings herein, the mobility-dependent analysis technique (MDAT) can comprise electrophoresis. Each mobility probe can include a detectable marker attached to, or otherwise associated with it; e.g., a fluorescent dye can be attached to each mobility probe. FIG. 6 illustrates components that can be included in various embodiments of a system. For example, a system can include a component for effecting a mobility dependent analysis technique, such as an electrophoresis instrument (e.g., a single- or multi-capillary sequencer) and a fluorescence detection unit, such as one or more photodiodes and/or CCDs (and associated optics, as desired), adapted to produce data signals to be analyzed in accordance with the teachings herein. It will be understood that suitable interfaces between the separate components, e.g., to adapt them for the transfer of information between the units, can be included.

In various embodiments, a sample can include one or more released mobility probes and, optionally, one or more sizing standards. For example, in various embodiments, a sample can include a plurality of released mobility probes, in accordance with the teachings herein, and a sizing standard comprised of a predetermined set of reference mobility probes designed to provide, in electrophoresis, a series of features (e.g., peaks) against which mobility data obtained for the released mobility probes can be analyzed.

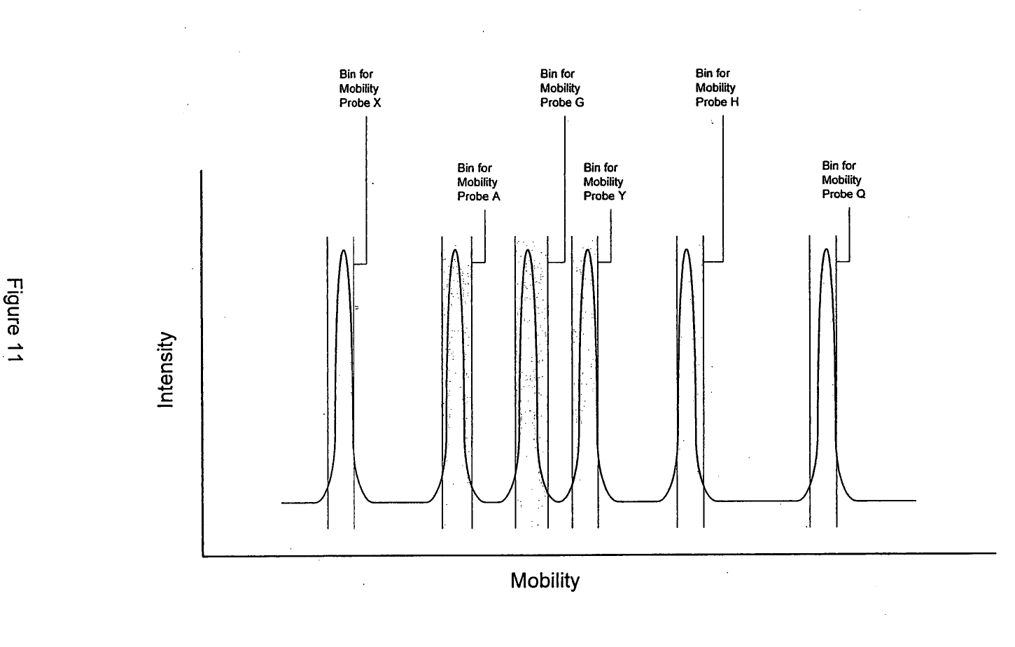

In some embodiments, the size standard can be used to define bins. A bin is a zone defined by the size standard that indicates where peaks would be expected to appear when a sample is run. In some embodiments, running the size standard results in a peak for every possible sample peak. In other embodiments, the size standard defines only some bins and other bins are inferred. FIG. 11 illustrates exemplary bins. Here, the size standard has been run on an electrophoresis instrument. The data shows peaks that correspond to the components of the size standard. Bins are indicated by the gray regions.

In an exemplary embodiment, and with reference to FIG. 6, a sample 103, prepared for electrophoresis and fluorescence detection and containing released mobility probes and a sizing standard, can be loaded onto an electrophoresis instrument 107 for separation into components or sample zones. The sample 103 can comprise, for example, mobility probes that have been released from respective targets or ligation products, as described herein, and a sizing standard comprising a preselected set of reference mobility probes. During or after the separation, the sample zones comprising the released mobility probes and the reference mobility probes can be detected by fluorescence emitted in response to excitation by an excitation source; e.g., laser beam or other light. It will be appreciated that the released mobility probes and the reference mobility probes can be associated with different fluorophores to facilitate distinguishing released mobility probes from reference mobility probes.

The fluorescence detection unit 109 can be adapted to produce signals 111, representing intensity levels of fluorescence for the various sample zones. The intensity signals 111 can be output or passed to a mobility-identification unit (MIU) 113, and, optionally, can be sent to an output and/or storage device, such as a display device (monitor) 117, a printer and/or disk drive, or the like. One skilled in the art will appreciate that various instrument and computer environments such as those described in U.S. patent application Ser. No. 09/658161 can be utilized with the present teachings, which application is incorporated herein by reference in its entirety for all purposes.

The mobility-identification unit 113, according to various embodiments, can interpret the intensity signals 111 and provide output corresponding to the identity, presence and/or absence of one or more target biochemicals and/or biochemical complexes of interest. For example, the mobility-identification unit can be adapted to identify in the output resulting from the mobility-dependent analysis technique, one or more features (e.g., peaks and/or characteristics thereof, such as height, area, etc.) that correspond to the mobility probes and, further, to associate the presence of said feature(s) with a particular target biochemical or biochemical complex of interest.

Mobility Identification Unit

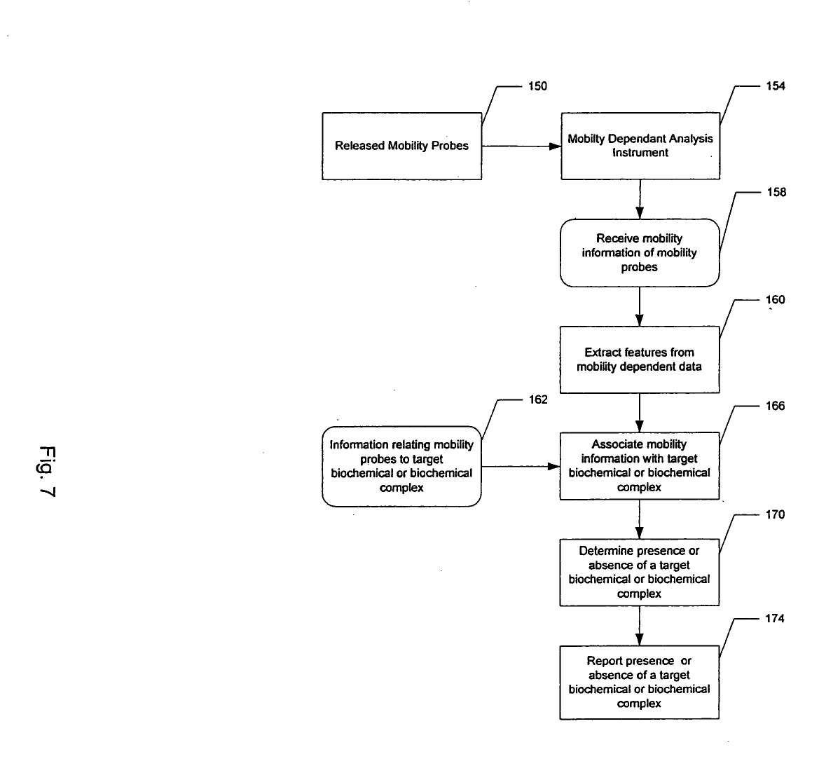

As previously indicated, in some embodiments, once the mobility probes have been released, they can be analyzed via a mobility-dependent analysis technique. An exemplary process is illustrated in FIG. 7. Released mobility probes (150) can undergo analysis in a mobility-dependent analysis instrument (154) whose output is mobility-dependent data. This data can be passed onto a system for further processing (158). The data can be factored into a set of features related to the probes (160). From the features, the presence or absence of particular mobility probes can be determined. From the presence or absence of the mobility probes, in turn, the presence or absence of target biochemicals or biochemical complexes can be ascertained. To accomplish this, for example, information that relates a given mobility probe to a particular target biochemical or biochemical complex (162) can be received and used to associate the features of the mobility dependent analysis with the mobility probes and, subsequently, with the target biochemical or biochemical complex. The presence or absence of the features extracted from the mobility dependent data thus decodes for the presence or absence of the target biochemical of biochemical complex (170). The results can then be reported (174).

Detection of Polymorphisms

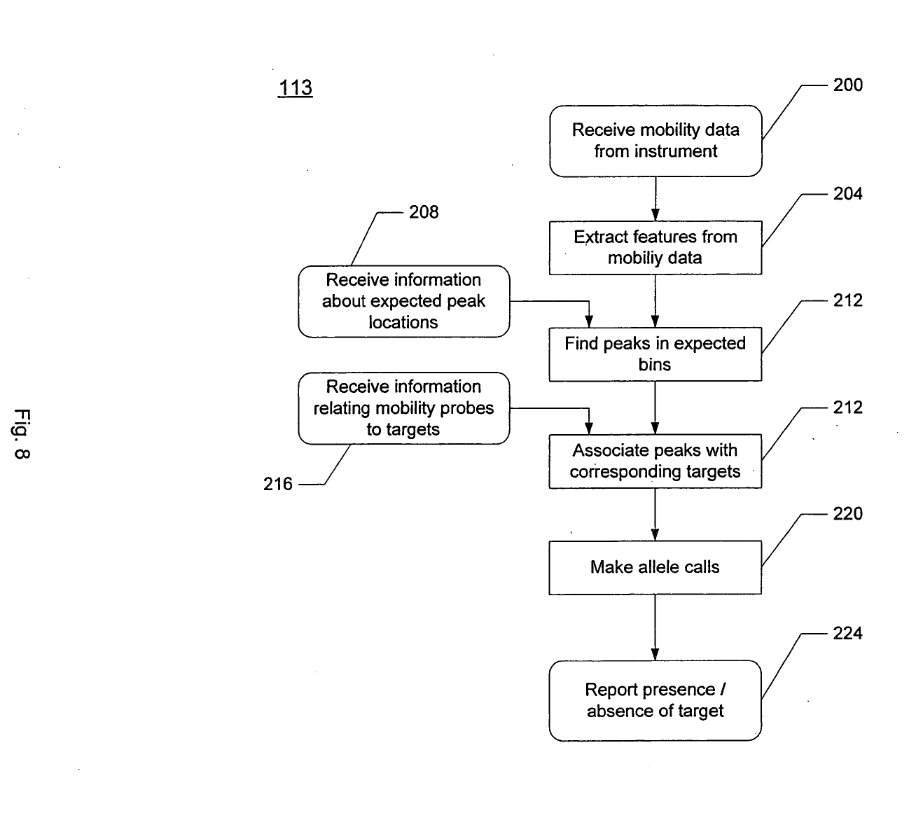

FIG. 8 illustrates an embodiment of a system that uses an electrophoresis instrument as the mobility-dependent analysis instrument and identifies bi-allelic single nucleotide polymorphisms (SNPs). Here, the mobility data is received from the electrophoresis instrument, for example, in the form of an electropherogram. The features of interest can be peaks that reflect the mobility and fluorescence intensity of the probes. Because the mobility probes are predefined, the positions of expected peaks corresponding to the mobility probes are known. This information can be used to retain only the peaks that relate to mobility probes, thereby eliminating from consideration at least some of any extraneous peaks or noise that may be present. Using the information that relates the mobility probes to the targets (216), the presence of the single nucleotide polymorphisms can be determined (220) and reported (224).

Allele Calling

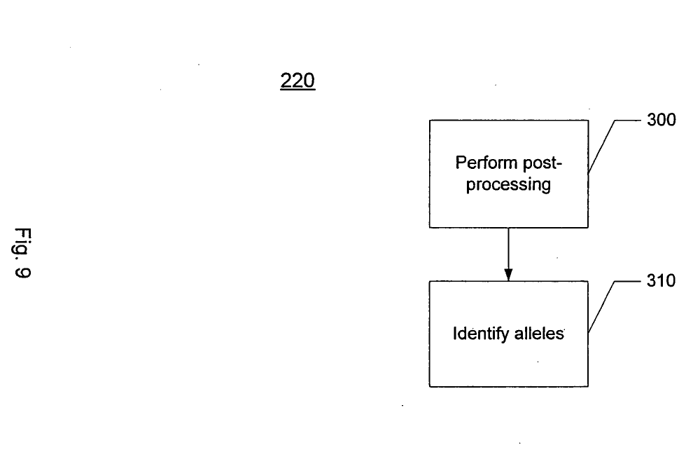

Various embodiments are contemplated for performing an allele-calling step or function, such as indicated at 220 in FIG. 8. An exemplary embodiment is illustrated in FIG. 9. In one embodiment of the system of FIG. 8, the presence or absence of a peak indicates the presence or absence of its corresponding mobility probe and hence target SNP. If a peak does not exist, the mobility probe and hence corresponding target are not present. In such a case, the post-processing referred to at (300) can be a pass-through function. It will be appreciated that contamination in a system, such as the system shown in FIG. 8, may result from accidental contamination by either mobility probes or other species that would cause a peak in one of the expected locations. This could compromise the results generated. To overcome this potential problem, and in accordance with various embodiments, the ratio of the height of the two peaks that are associated with a SNP can be computed.

$$R = \frac{peak height smallest peak}{peak height of largest peak}$$

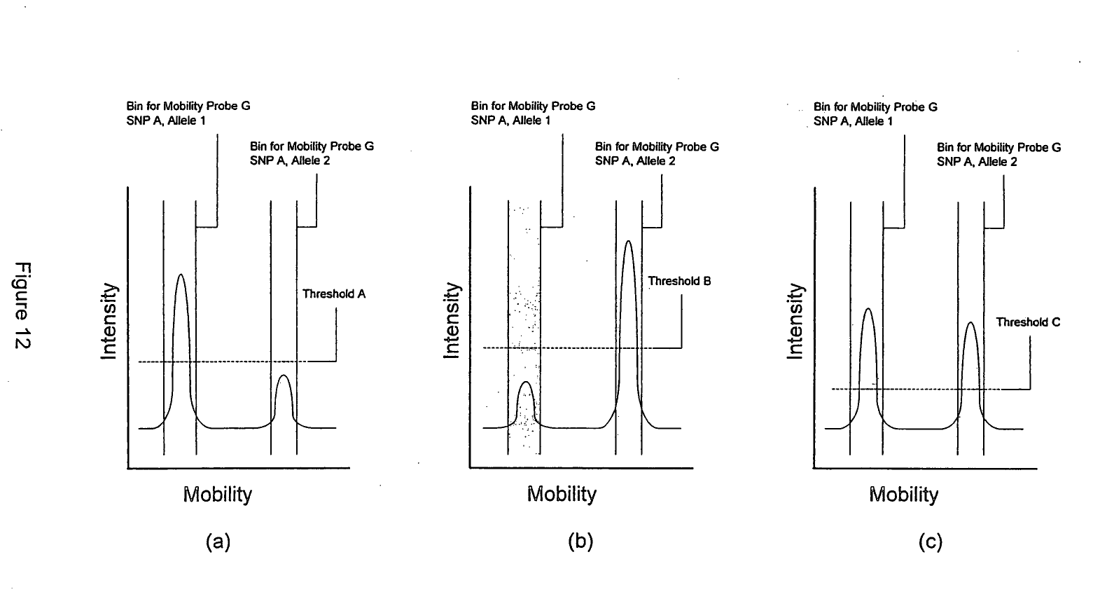

If the ratio of the lowest peak to the highest peak is less than some selected threshold (e.g., 2/3; 1/2; 1/3; or 1/4), the SNP is said to be homozygous for the allele with the higher peak. Otherwise, the sample is said to be heterozygous. FIG. 12 illustrates an embodiment of such a system. In FIG. 12(a) the ratio of the peak height of the smaller peak (which is associated with allele 2 of a single nucleotide polymorphism called A), to the bigger peak (which is associated with allele 1 of a single nucleotide polymorphism called A) does not exceed a threshold (Threshold B) hence this sample would be called homozygous for allele 1. Similarly, in FIG. 12(b) the ratio of the peak height of the smaller peak (which is associated with allele 1 of a single nucleotide polymorphism called A), to the bigger peak (which is associated with allele 2 of a single nucleotide polymorphism called A) does not exceed a threshold (Threshold B) hence this sample would be called homozygous for allele 2. Finally, in FIG. 12(c), the ratio of the peak height of the smaller peak (which is associated with allele 2 of a single nucleotide polymorphism called A), to the bigger peak (which is associated with allele 1 of a single nucleotide polymorphism called A) does not exceed a threshold (Threshold C) hence this sample would be called heterozygous for allele 2. The same principle can be extended to tri-allelic SNPS.

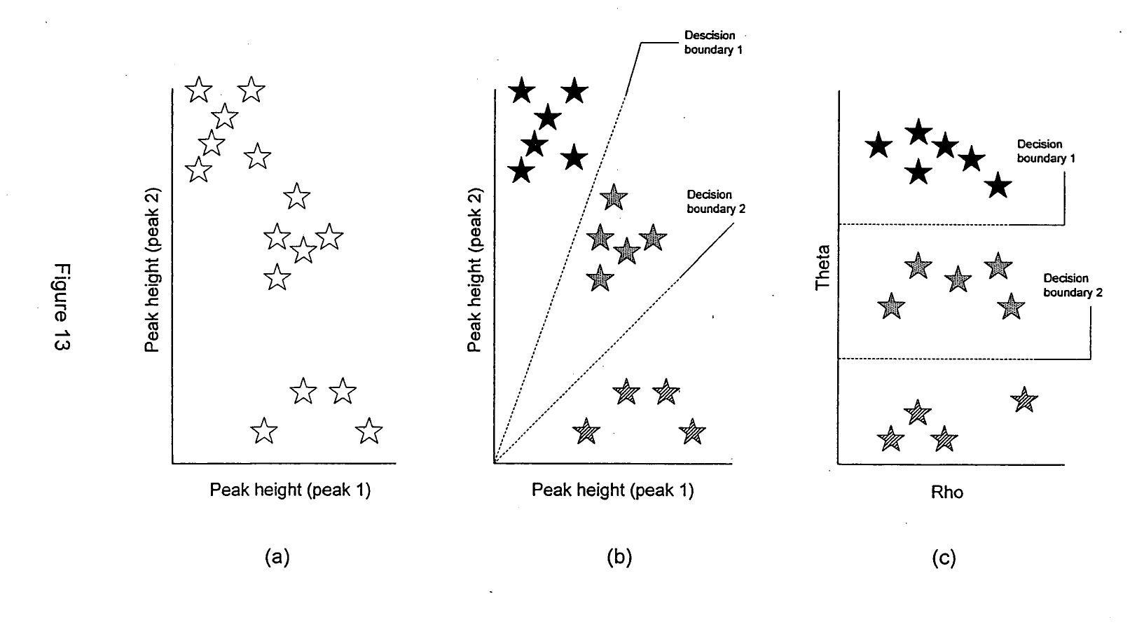

Various embodiments of an allele calling system can use clustering. This can be useful, for example, when several samples are to be analyzed at once. An exemplary clustering process is illustrated in FIG. 13. In FIG. 13(a), data points are represented by stars that are plotted in a Cartesian system according to their attributes of peak heights. The clustering mechanism serves to assign each point a group membership as shown in FIG. 13(b). One skilled in the art will appreciate that certain data transformations can facilitate the process of clustering. FIG. 13(c) shows a conversion of the data used in FIG. 13(b) into polar coordinates. Here the clusters are imparted with better separation.

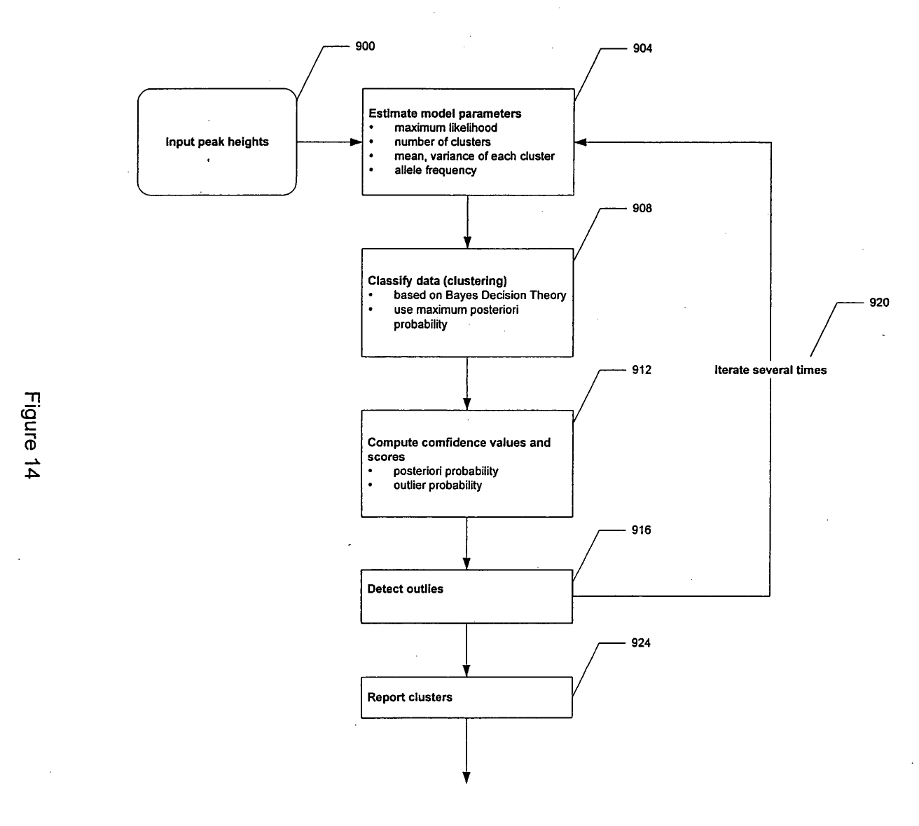

In various embodiments, data points can each be assigned a set of attributes and a similarity metric can be calculated based on those attributes. This metric relates each data point to each other data point. The process of clustering can thereby serve to find clusters such that data points in one cluster are more similar to one another and data points in separate clusters are less similar to one another. Confidence values that dictate a reasonable confidence that a data point belongs to the assigned cluster can be computed based on the metrics used to define the clusters. In other various embodiments of clustering, a priori information is built into a model. This information, in addition to the attributes assigned to the data points, can be used to form clusters with the aforementioned properties. An embodiment of clustering can include the use of the Maximum Likelihood algorithm to compute the cluster memberships in an optimal way. Confidence values can be calculated based on the model fit and the metrics used to define the clusters. An embodiment of this is illustrated in FIG. 14. This figure illustrates an iterative process. Data attributes are fed into the system (902) and the model parameters are computed. Some embodiments use the number of clusters, mean and variance of each cluster and the expected number of data points in each cluster (step 904) in the model. In step 908, the points are assigned to clusters using the a posteriori probability. This is the probability of a given data point belonging to a given cluster. When the statistical model is estimated, the a posteriori probability can be calculated using Bayes formula. The a posteriori probability is a useful concept in Bayes decision theory as described in many textbooks such as in reference [1], incorporated herein by reference. In step 912, confidence values can be computed for each point using one or more assumed probabilities. These can include one or more of the model fit probability, which estimates the confidence of the estimated model, the a posteriori probability, which states that given the estimated model, the probability that a given point belongs to an assigned cluster and the outlier probability, which estimates the probability that the cluster could produce a given sample point. In 916, outliers can be detected. The in-class probability is a measure of the probability that a given point is produced from the assigned cluster given the estimated model. The model fitting and cluster assignment process can be repeated (step 920) until some specified accuracy is obtained at which point the clusters are reported. Aspects of such a system can be found in U.S. Provisional Patent Application 60/392841 filed Jun. 20, 2002, which application is incorporated herein by reference in its entirety for all purposes.

Various of the functions described herein, e.g., bin building, allele calling, clustering, etc., can be performed by methods utilized in the GENEMAPPER Software and the SNP MANAGER Software, available from Applied Biosystems (Foster City, Calif.). See, for example, the “ABI PRISM® GeneMapper Software Version 3.0 User's Manual” and the “SNP example, the “ABI PRISM® GeneMapper Software Version 3.0 User's Manual” and the “SNP See also U.S. patent applications Ser. No. 60/227556, filed Aug. 23, 2000; Ser. No. 60/290129, filed May 10, 2001; Ser. No. 09/724910, filed November 28, 2000; and Ser. No. 09/911903, filed Jul. 23, 2001; expressly incorporated herein by reference in their entireties.

Computer Implementation

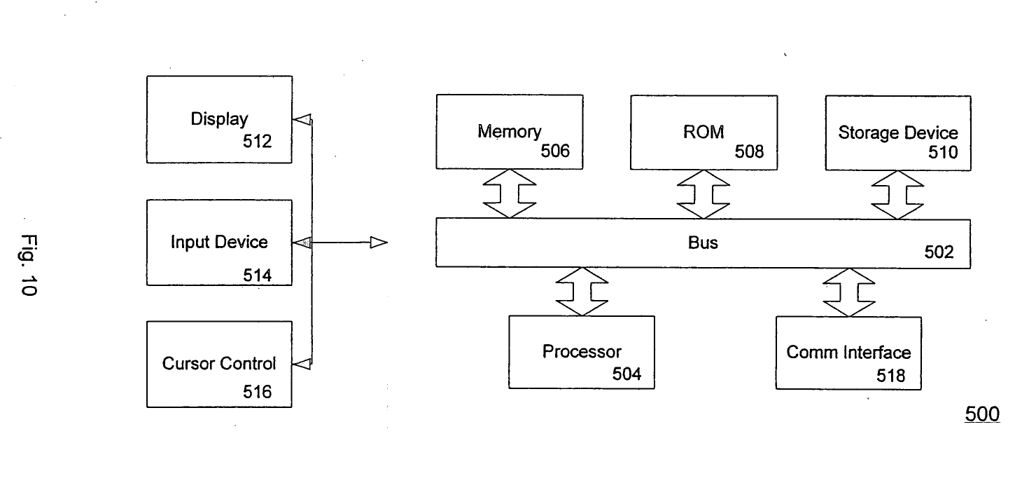

FIG. 10 is a block diagram that illustrates a computer system 500, according to certain embodiments, upon which embodiments of the invention may be implemented. Computer system 500 includes a bus 502 or other communication mechanism for communicating information, and a processor 504 coupled with bus 502 for processing information. Computer system 500 also includes a memory 506, which can be a random access memory (RAM) or other dynamic storage device, coupled to bus 502 for determining base calls, and instructions to be executed by processor 504. Memory 506 also may be used for storing temporary variables or other intermediate information during execution of instructions to be executed by processor 504. Computer system 500 further includes a read only memory (ROM) 508 or other static storage device coupled to bus 502 for storing static information and instructions for processor 504. A storage device 510, such as a magnetic disk or optical disk, is provided and coupled to bus 502 for storing information and instructions.

Computer system 500 may be coupled via bus 502 to a display 512, such as a cathode ray tube (CRT) or liquid crystal display (LCD), for displaying information to a computer user. An input device 514, including alphanumeric and other keys, is coupled to bus 502 for communicating information and command selections to processor 504. Another type of user input device is cursor control 516, such as a mouse, a trackball or cursor direction keys for communicating direction information and command selections to processor 504 and for controlling cursor movement on display 512. This input device typically has two degrees of freedom in two axes, a first axis (e.g., x) and a second axis (e.g., y), that allows the device to specify positions in a plane.

A base or allele call is provided by computer system 500 in response to processor 504 executing one or more sequences of one or more instructions contained in memory 506. Such instructions may be read into memory 506 from another computer-readable medium, such as storage device 510. Execution of the sequences of instructions contained in memory 506 causes processor 504 to perform the process states described herein. Alternatively hard-wired circuitry may be used in place of or in combination with software instructions to implement the invention. Thus implementations of the invention are not limited to any specific combination of hardware circuitry and software.

The term “computer-readable medium” as used herein refers to any media that participates in providing instructions to processor 504 for execution. Such a medium may take many forms, including but not limited to, non-volatile media, volatile media, and transmission media. Non-volatile media includes, for example, optical or magnetic disks, such as storage device 510. Volatile media includes dynamic memory, such as memory 506. Transmission media includes coaxial cables, copper wire, and fiber optics, including the wires that comprise bus 502. Transmission media can also take the form of acoustic or light waves, such as those generated during radio-wave and infra-red data communications.

Common forms of computer-readable media include, for example, a floppy disk, a flexible disk, hard disk, magnetic tape, or any other magnetic medium, a CD-ROM, any other optical medium, punch cards, papertape, any other physical medium with patterns of holes, a RAM, PROM, and EPROM, a FLASH-EPROM, any other memory chip or cartridge, a carrier wave as described hereinafter, or any other medium from which a computer can read.

Various forms of computer readable media may be involved in carrying one or more sequences of one or more instructions to processor 504 for execution. For example, the instructions may initially be carried on magnetic disk of a remote computer. The remote computer can load the instructions into its dynamic memory and send the instructions over a telephone line using a modem. A modem local to computer system 500 can receive the data on the telephone line and use an infra-red transmitter to convert the data to an infra-red signal. An infra-red detector coupled to bus 502 can receive the data carried in the infra-red signal and place the data on bus 502. Bus 502 carries the data to memory 506, from which processor 504 retrieves and executes the instructions. The instructions received by memory 506 may optionally be stored on storage device 510 either before or after execution by processor 504.

### EXAMPLE

In a non-limiting example, a method according to the present teachings can comprise one or more of the following:
1) receiving an electropherogram (e.g., from a capillary-type electrophoresis instrument);
2) extracting features (e.g., peaks and/or characteristics thereof);
3) associating the features (e.g., peaks) with respective mobility probes;
4) associating the mobility probes with respective targets;
5) performing one or both of (a) a ratio step or (b) a clustering step to determine if the features/mobility probe association do represent the presence of the target (note: as some peaks could be due to poor wash steps etc,).
   Steps 1) through 5) can be carried out a plurality of times, in series or in parallel. The results of step 5), for each iteration, can be reported and/or entered into a database.

All references cited herein are incorporated by reference for any purpose as if each was separately but expressly incorporated by reference.

Although the invention has been described with reference to various embodiments, it will be appreciated that various changes and modifications may be made without departing from the scope and spirit of the present teachings.

It is claimed:

1. A method for determining the presence or absence of a target biochemical or biochemical complex in a sample, comprising:
(i) receiving mobility data from an analysis of one or more released mobility probes using a mobility dependent analysis technique;
(ii) extracting at least one feature or feature set from the mobility data;
(iii) receiving information for associating the at least one feature or feature set to the one or more released mobility probes;
(iv) associating one of the one or more released mobility probes with a respective target biochemical or biochemical complex; and
(v) determining the presence or absence of the target biochemical or biochemical complex.

2. The method of claim 1, further comprising:
reporting the presence or absence of the target biochemical or biochemical complex.

3. The method of claim 1, wherein the mobility dependent analysis technique comprises electrophoresis.

4. The method of claim 1, wherein the target biochemical or biochemical complex comprises a nucleic acid, protein, or peptide.

5. The method of claim 1, wherein the target biochemical or biochemical complex comprises a single nucleotide polymorphism.

6. The method of claim 5, wherein the at least one feature or feature set comprises a plurality of peaks, and further wherein the step of determining the presence or absence of the target biochemical or biochemical complex comprises:
computing a ratio of smallest peak height to largest peak height for a set of two peaks associated with the single nucleotide polymorphism; and
calling the single nucleotide polymorphism heterozygous if the ratio is greater than a selected threshold.

7. The method of claim 5, wherein the determination of the presence or absence of a target biochemical or biochemical complex includes utilization of a clustering technique.

8. A method for determining the presence or absence of a target biochemical or biochemical complex in a sample, comprising:
(i) receiving mobility data from an analysis of a plurality of released mobility probes using a mobility dependent analysis technique;
(ii) extracting a feature set from the mobility data;
(iii) receiving information for associating the feature set to the plurality of released mobility probes;
(iv) associating a released mobility probe with a respective target biochemical or biochemical complex;
(v) determining the presence or absence of the target biochemical or biochemical complex.

9. The method of claim 8, further comprising:
reporting the presence or absence of the target biochemical or biochemical complex.

10. The method of claim 8, further comprising:
entering the presence or absence of the target biochemical or biochemical complex into a database.

11. A program storage device readable by a machine, embodying a program of instructions executable by the machine to perform method steps for analysis of a target biochemical or biochemical complex in a sample, said method steps comprising:
(i) receiving mobility data from an analysis of one or more released mobility probes using a mobility dependent analysis technique;
(ii) extracting at least one feature or feature set from the mobility data;
(iii) receiving information for associating the at least one feature or feature set to the one or more released mobility probes;
(iv) associating one of the one or more released mobility probes with a respective target biochemical or biochemical complex; and
(v) determining the presence or absence of the target biochemical or biochemical complex.

12. The device of claim 11, wherein said method steps further comprise:
reporting the presence or absence of the target biochemical or biochemical complex.

13. The device of claim 11, wherein said method steps further comprise:
entering the presence or absence of the target biochemical or biochemical complex in a database.

14. A method for genetic analysis, comprising:
analyzing a plurality of samples on an electrophoresis instrument, with each sample representing an individual of a population, whereby mobility data is generated for each sample;
receiving said mobility data, represented as fluorescence intensity over time;
associating the mobility data with the presence or absence of one or more target biochemicals or biochemical complexes in the samples;
transforming the mobility data to a different feature space;
assigning a class to each sample based on said transforming; and
determining a genoptypic characteristic of each sample or the population based the class assignment.

15. The method of claim 14, wherein the step of transforming the data to a different feature space includes transformation to rho theta coordinates.

16. The method of claim 14, wherein the step of assigning a class to each sample includes utilization of a clustering technique.

17. A program storage device readable by a machine, embodying a program of instructions executable by the machine to perform genetic analysis, said method steps comprising:
receiving mobility data, represented as fluorescence intensity over time, generated by electrophoresis of a plurality of samples, with each sample representing an individual of a population;
associating the mobility data with the presence or absence of one or more target biochemicals or biochemical complexes in the samples;
transforming the mobility data to a different feature space; and
assigning a class to each sample based on said transforming.

18. The device of claim 17, wherein said method steps further comprise:
determining a genoptypic characteristic of each sample or the population based the class assignment.

19. A method for determining a target biochemical or biochemical complex, comprising:
(i) receiving mobility-dependent data representing the output of a mobility-dependent analysis technique;
(ii) receiving data for associating the mobility-dependent data with a mobility probe;
(iii) receiving data for associating the mobility probe with a tag or a complimentary tag sequence;
(iv) receiving data for associating the tag or complimentary tag sequence with a target;
and, using the data from (ii), (iii), and (iv):
(v) associating the mobility-dependent data with a corresponding mobility probe;
(vi) associating the mobility probe from (v) with a corresponding tag or complimentary tag sequence;
(vii) associating the tag or complimentary tag sequence from (vi) with a corresponding target; and
(viii) reporting the detection of the target from (vii).

20. The method of claim 19, wherein steps (i) through (viii) are performed two or more times, in a serial fashion.

21. The method of claim 19, wherein steps (i) through (viii) are performed two or more times, in a parallel fashion.

22. A method for determining a target biochemical or biochemical complex, comprising:
(i) receiving mobility-dependent data representing the output of a mobility- dependent analysis technique;
(ii) receiving data for associating the mobility-dependent data with a plurality of mobility probes;
(iii) receiving data for associating each of the mobility probes with a respective tag or complimentary tag sequence;
(iv) receiving data for associating the tag or complimentary tag sequence from (iii) with a respective target;
and, using the data from (ii), (iii), and (iv):
(v) associating the mobility-dependent data with the plurality of mobility probes;
(vi) associating each of the plurality of mobility probes with a corresponding tag or complimentary tag sequence;
(vii) associating each tag or complimentary tag sequence from (vi) with a corresponding target; and
(viii) reporting the detection of each target from (vii).

23. The method of claim 22, wherein step (viii) includes entering the detection of each target into a database.

24. A program storage device readable by a machine, embodying a program of instructions executable by the machine for determining a target biochemical or biochemical complex, said method steps comprising:
(i) receiving mobility-dependent data representing the output of a mobility-dependent analysis technique;
(ii) receiving data for associating the mobility-dependent data with a mobility probe;
(iii) receiving data for associating the mobility probe with a tag or a complimentary tag sequence;
(iv) receiving data for associating the tag or complimentary tag sequence with a target;
and, using the data from (ii), (iii), and (iv):
(v) associating the mobility-dependent data with a corresponding mobility probe;
(vi) associating the mobility probe from (v) with a corresponding tag or complimentary tag sequence;
(vii) associating the tag or complimentary tag sequence from (vi) with a corresponding target; and
(viii) reporting the detection of the target from (vii).

25. A program storage device readable by a machine, embodying a program of instructions executable by the machine for determining one or more target biochemicals or biochemical complexes, said method steps comprising:
(i) receiving mobility-dependent data representing the output of a mobility- dependent analysis technique;
(ii) receiving data for associating the mobility-dependent data with a plurality of mobility probes;
(iii) receiving data for associating each of the plurality of mobility probes with a respective target biochemical or biochemical complex;
and, using the data from (ii) and (iii):
(iv) associating the mobility-dependent data with the mobility probes;
(v) associating each of the mobility probes from (iv) with a corresponding target; and
(vi) reporting the detection of the target from (v).

26. The device of claim 25, wherein the mobility probes are released mobility probes.

27. A method for genetic analysis, comprising:
receiving electropherogram data;
extracting peaks from the electropherogram data;
associating the peaks with respective released mobility probes;
associating the released mobility probes with respective targets;
analyzing the peaks against selected criteria to filter out peaks not indicative of targets; and
reporting the detection of targets.
